(19) United States
(12) Patent Application Publication
(10) Pub. No.: US 2025/0032123 A1
(43) Pub. Date: Jan. 30, 2025

(54) ANCHORS FOR A COLLAPSING IMPLANT

(71) Applicant: Endomatic Ltd., Or Yehuda (IL)

(72) Inventors: Dvir KEREN, Tel Aviv (IL); Boaz SCHWARZ, Tel-Aviv (IL); Shimrit MARKOVITZ, Tel Aviv (IL); Lior SHAHORY, Herzliya (IL)

(73) Assignee: Endomatic Ltd., Or Yehuda (IL)

(21) Appl. No.: 18/715,765

(22) PCT Filed: Dec. 7, 2022
(86) PCT No.: PCT/IL2022/051299

§ 371 Date: Jun. 3, 2024

Related U.S. Application Data
(60) Provisional application No. 63/286,644, filed on Dec. 7, 2021.

(51) Int. Cl. A61B 17/12; A61B 17/00

(52) U.S. Cl. CPC A61B 17/12122; A61B 2017/00579; A61B 2017/00592; A61B 2017/00628; A61B 2017/00632

(57) ABSTRACT

Anchoring for arms of a suture clip. In some embodiments, an arm-attached anchoring element undergoes a change in shape as it is released from overtube confinement, re-orienting its sharpened tip so that it is prepared for insertion into tissue. The anchoring element may be shaped with recesses and elastic members positioned so that the anchoring element resists deformation strongly once released, but resists less forcefully while held in its confined shape. In some embodiments, an anchoring element is constructed of a first piece, and an arm is constructed as part of a second piece. Arm and anchoring element are assembled, for example, using interlocking shapes cut into each.

### RELATED APPLICATIONS

This application claims the benefit of priority under 35 USC § 119(e) of U.S. Provisional Patent Application No. 63/286,644 filed Dec. 7, 2021; the contents of which are incorporated herein by reference in their entirety.

### FIELD AND BACKGROUND OF THE INVENTION

The present invention, in some embodiments thereof, relates to the field of closure devices for medical use, and more particularly, to closure devices for use in the LAA.

The medical community considers the left atrium appendage (LAA) as a potential causal locus for CVA (cardiovascular accident) due to the potential of the LAA for embolic creation.

Closing off the LAA may be performed, for example, in an open thoracic approach or by a minimally invasive trans-vascular (and typically trans-septal) approach. In the open approach, a surgeon is likely to suture the ostium (the connection between the LAA and the left atrium). In a trans-vascular approach, an interventional cardiologist doesn't open the patient's chest, and seals the LAA, e.g., using a plug and/or stent-like construction deployed inside the LAA. The principle is that reduction of atrial wall irregularities and/or circulatory dead zones may reduce a potential for thrombogenesis.

### SUMMARY OF THE INVENTION

According to an aspect of some embodiments of the present disclosure, there is provided a self-expanding tissue anchoring mechanism configured to deploy from an arm of an implantable device, the anchoring mechanism including: an arm, including an anchor-mounting portion; an anchoring element having a sharp distal end, and a proximal end engaged with the anchor-mounting portion; wherein, together, the proximal end and anchor-mounting portion form an assembly, the assembly having an elastic member which relaxes to interconvert the assembly between a first shape and a second shape; and the first shape is collapsed to position a distal portion of the sharp distal end of the anchoring element relatively close to a longitudinal axis of the arm, compared to the second shape.

According to some embodiments of the present disclosure, the elastic member is tensioned in the first shape, and relaxed in the second shape.

According to some embodiments of the present disclosure, interconversion between the first shape and the second shape moves the distal portion of the sharp distal end of the anchoring element longitudinally along the longitudinal axis, and further away from the longitudinal axis.

According to some embodiments of the present disclosure, the elastic member includes a spring formed into the anchoring element.

According to some embodiments of the present disclosure, the anchoring element includes a piece cut from sheet stock, and the elastic member is a part of the piece.

According to some embodiments of the present disclosure, the anchoring element and the anchor-mounting portion comprise separate interlocking pieces.

According to some embodiments of the present disclosure, the anchoring element includes: a base, attached to the anchor-mounting portion of the arm; and the elastic member, extending between the sharp distal end and the base.

According to some embodiments of the present disclosure, the anchoring element includes: a base, attached in a plurality of separate base regions to the anchor-mounting portion of the arm; wherein the elastic member joins the sharp distal end to one of the base regions; and an elongated member joining the sharp distal end to another of the base regions, bypassing the elastic member.

According to some embodiments of the present disclosure, the implantable device is a suturing clip including a plurality of arms extending from a core, each provided with a corresponding anchor element.

According to some embodiments of the present disclosure, the sharp distal end is held away from the anchor mounting portion of the arm by an elongated member, wherein a longitudinal axis of the elongated member extends away from a longitudinal axis of the arm at an approximate right angle in the second shape, and at an oblique angle in the first shape which is at least 30° different than the approximate right angle shape.

According to some embodiments of the present disclosure, the oblique angle in the first shape is at least 45° different than the approximate right angle shape.

According to some embodiments of the present disclosure, the elastic member of the assembly is part of the proximal end of the anchoring element.

According to some embodiments of the present disclosure, the elastic member terminates in a recess shaped to engage with the anchor-mounting portion.

According to some embodiments of the present disclosure, the elastic member of the assembly is part of the anchor-mounting portion.

According to some embodiments of the present disclosure, the elastic member terminates in a recess shaped to engage with the anchoring element.

According to an aspect of some embodiments of the present disclosure, there is provided a self-expanding tissue anchoring mechanism configured to deploy from an arm of an implantable device, the anchoring mechanism including: an anchoring element terminating in a sharp distal end and attached, through a plurality of base regions on its proximal side, to an anchor-mounting portion of the arm; wherein the sharp distal end is joined to a first of the plurality of base regions through an elastic member, and separately to a second of the plurality of base regions through a separate elongated member.

According to some embodiments of the present disclosure, the elastic member bends, moving the sharp distal end from a collapsed position nearer to a longitudinal axis of the anchor-mounting portion of the arm, to an expanded position further from the longitudinal axis.

According to some embodiments of the present disclosure, the second of the base regions includes an open-sided recess, the recess being engaged with the anchor-mounting portion of the arm when the sharp distal end is in the expanded position, and disengaged when the sharp distal end is in the collapsed position.

According to some embodiments of the present disclosure, the open-sided recess is positioned near a base end of an elongated member, and as the sharp distal end moves from the collapsed position to the expanded position, the elongated member slides along the anchor-mounting portion of the arm until the open-sided recess engages with the anchor-mounting portion.

According to some embodiments of the present disclosure, the first of the plurality of base regions includes a pair of members which grasp a portion of the anchor-mounting portion of the arm from on opposite sides of the anchor-mounting portion.

According to some embodiments of the present disclosure, the pair of members clasp around a bar to define a slot between them through which the bar is passed to snap-fit the anchoring element to the anchor-mounting portion of the arm.

According to an aspect of some embodiments of the present disclosure, there is provided a suturing clip including a plurality of arms joined to a common core, and a respective anchoring element in an interlocking attachment with a distal end of each respective arm, the anchoring element being provided with a sharpened distal tip, a barb, and an elongated member which attaches on a proximal side to the arm.

According to some embodiments of the present disclosure, the anchor includes a tubular member mounted through an aperture of the arm.

According to some embodiments of the present disclosure, the tubular member is sharpened at its tip, and cut along its length to define a barb.

According to some embodiments of the present disclosure, the suturing clip includes a pin which passes through apertures in both the anchor and the arm to hold them together, and a plate having an aperture which passes over both the anchor and the arm.

According to an aspect of some embodiments of the present disclosure, there is provided a method of expanding an anchoring member from an arm of a suturing clip, the method including: providing an anchoring member attached to a portion of the arm, and confined within a lumen of an overtube, wherein the anchoring member includes an elongated member: contacting the arm at a proximal end of the elongated member, terminating in a distal sharpened end, and constrained by confinement of the anchoring member to an oblique angle with respect to a longitudinal axis of the portion of the arm; releasing the anchoring member from confinement within the lumen; and as the anchoring member is released from confinement within the lumen: sliding the elongated member along the arm until a recess of the elongated member reaches the arm and grasps it, and pivoting the elongated member around a region of contact between the arm and the recess until the elongated member reaches an approximate right angle with respect to the longitudinal axis.

According to some embodiments of the present disclosure, the sliding and the pivoting are driven by tension released from a spring member as the anchoring member is released from confinement within the lumen.

According to some embodiments of the present disclosure, the sliding advances the elongated member through an aperture of the arm.

According to some embodiments of the present disclosure, the pivoting rotates the recess around a wall of the aperture which is within the grasp of the recess.

According to an aspect of some embodiments of the present disclosure, there is provided a method of collapsing an anchoring member on an arm of a suturing clip, the method including: providing an anchoring member attached to a portion of the arm protruding from a lumen of an overtube, wherein the anchoring member includes an elongated member: having a recess of the anchoring member located at a proximal end of the elongated member where the recess is positioned to grasp a region of the arm, and terminating in a distal sharpened end; withdrawing the arm and the attached anchoring member into the lumen; and as the anchoring member is withdrawn into lumen: under contact force exerted by the overtube, pivoting the elongated member around a region of contact between the arm and the recess until the recess releases from engagement with the region of the arm, and sliding the elongated member further along the arm; thereby flattening the elongated member into a more oblique angle with respect to a longitudinal axis of the portion of the arm to which it attaches.

According to some embodiments of the present disclosure, the sliding and the pivoting develop tension in a spring member of the anchoring member.

According to some embodiments of the present disclosure, the anchoring member is embedded in tissue, and including extracting the anchoring member from tissue of less than 5 mm thickness without leaving behind an open hole.

According to an aspect of some embodiments of the present disclosure, there is provided an ablation device sized to insert into an ostium of an LAA, and including a plurality of arms extending from a common core, each arm being electrically insulated and terminating in a non-insulated contact region, the ablation device being configured to be coupled to a power source to receive radio frequency (RF) power therefrom; wherein, while the device is positioned at the LAA ostium, the non-insulated contact regions are moved by expansion of the arms to positions where they contact tissue around the circumference of the LAA, allowing delivery of RF power to the 15 device to ablate the contacted tissue.

According to an aspect of some embodiments of the present disclosure, there is provided a method of positioning a distal portion of a deployment system including a suturing clip within an ostium of a LAA of a heart, in preparation for deploying the suturing clip, the method including: rotating the suturing clip to a predetermined orientation relative to the ostium; centering the distal portion of the deployment system between posterior and anterior walls of the ostium; and advancing the distal portion of the deployment system by about 1-8 mm beyond a circumflex coronary artery of the heart.

According to some embodiments of the present disclosure, the rotating is performed under fluoroscopic visualization; and the centering and advancing are performed under ultrasound visualization.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A schematically illustrates a suturing clip engaged to tissue within a LAA, according to some embodiments of the present disclosure;

FIG. 1B schematically illustrates the suturing clip of FIG. 1A in an expanded configuration suitable for tissue engagement, according to some embodiments of the present disclosure;

FIG. 1C schematically illustrates a suturing clip which has engaged tissue within an LAA and then undergone a conformational change to close an LAA ostium, according to some embodiments of the present disclosure;

FIG. 1D schematically illustrates the suturing clip of in the LAA-closing conformation of FIG. 1C, according to some embodiments of the present disclosure;

FIG. 1E schematically illustrates a suturing clip confined to a delivery configuration by an overtube, according to some embodiments of the present disclosure;

FIG. 1F is a schematic flowchart of operations related to an anchor of a suturing clip, according to some embodiments of the present disclosure;

FIGS. 1G-1I together schematically illustrate a method of expanding, and optionally re-collapsing, an anchor of a suture clip, according to some embodiments of the present disclosure;

FIGS. 2A-2D schematically illustrate stages in the deployment of a suturing clip, according to some embodiments of the present disclosure;

FIGS. 3A-3C schematically illustrate movements of anchoring positions over the course of a transition of a suturing clip between a deployed-and-anchored configuration, and a collapsed suturing configuration, according to some embodiments of the present disclosure;

FIGS. 4A-4C schematically represent views from different directions of a suturing clip in a partially deployed and fully arm-expanded configuration, according to some embodiments of the present disclosure;

FIGS. 5A-5C schematically represent view from different directions of suturing clip in a fully deployed collapsed suturing configuration, according to some embodiments of the present disclosure;

FIGS. 6A-6D schematically represent a sprung tissue anchoring mechanism, according to some embodiments of the present disclosure;

FIGS. 7A-7C schematically represent a variation of a sprung tissue anchoring mechanism, according to some embodiments of the present disclosure;

FIGS. 8A-8B show one branched arm of a suturing clip in a partially collapsed configuration (FIG. 8A) and a partially deployed configuration (FIG. 8B), according to some embodiments of the present disclosure;

FIGS. 9A-9E schematically illustrate a mechanism which provides releasable attachment between a suture clip and its delivery mechanism, according to some embodiments of the present disclosure;

FIGS. 10A-10C schematically illustrate a fixed-shape two-part anchor mechanism, according to some embodiments of the present disclosure;

FIGS. 11A-11B schematically illustrate a needle-based anchor mechanism, according to some embodiments of the present disclosure;

FIGS. 12A-12B schematically illustrate an anchoring member secured to an arm distal section by interlocking fasteners, according to some embodiments of the present disclosure;

FIG. 13 schematically represents internal structure of an overtube of a distal tip of a catheter for delivering a suturing clip, according to some embodiments of the present disclosure;

FIGS. 14A-14B show, respectively, a fluoroscopic image (FIG. 14A) and corresponding image trace-over (FIG. 14B) of a suturing clip and delivery system in situ as it passes through an LAA ostium, according to some embodiments of the present disclosure.

FIGS. 15A-15B show, respectively, a 3-D ultrasound image (FIG. 15A) and corresponding image trace-over (FIG. 15B) of a delivery system for a suturing clip in situ as it passes through an LAA ostium, according to some embodiments of the present disclosure; and

FIGS. 16A-16B show, respectively, a 2-D ultrasound image (FIG. 16A) and corresponding image trace-over (FIG. 16B) of a delivery system for a suturing clip in situ as it passes through an LAA ostium, according to some embodiments of the present disclosure.

### DESCRIPTION OF SPECIFIC EMBODIMENTS OF THE INVENTION

The present invention, in some embodiments thereof, relates to the field of closure devices for medical use, and more particularly, to closure devices for use in the LAA.

### Overview

### Collapsible Anchoring Mechanisms

An aspect of some embodiments of the present disclosure relates to tissue anchoring mechanisms of tissue closure devices, configured to transition from a collapsed delivery configuration to an expanded anchoring configuration, according to some embodiments of the present disclosure. In some embodiments, the tissue closure device is a closure clip for a left atrial appendage (LAA).

In some embodiments, the tissue closure device is delivered to an implantation region in a packaged configuration referred to herein as a delivery configuration. In the delivery configuration, the closure device is collapsed to fit within a tubular delivery sheath. After unsheathing, the tissue closure device expands to occupy a larger radial extent, comprising one or more expanded configurations, and optionally a staged sequence of expanded configurations (also referred to herein as “deployed” configurations, comprising one or more stages of deployment).

In some embodiments, more particularly, an LAA closure device—including anchoring elements—is delivered in a low-diameter configuration suitable for minimally invasive delivery; e.g., via catheter or other tubular delivery device. In some embodiments, delivery packaging of the device is preferably sized for transseptal delivery (delivery across the septal wall between the right atrium to the left atrium), and preferably sized to allow access to the heart itself via a transvascular pathway (entering, e.g., from the superior or inferior vena cava).

Device expansion may be at least partially controlled by an extent of unsheathing of the device (that is, expansion progresses through a series of configurations, according to an amount of unsheathing). For example, members of the device, in some embodiments, are constructed of a superelastic material such as nitinol. The members are shape-biased to assume a more expanded configuration when unconstrained, and also sized and arranged to allow confinement within the tubular delivery sheath.

In a packaged configuration, straight and stiff elements of the device are constrained to extend along a longitudinal axis of the package, or across (and not beyond) a diameter of the package. With more flexibility, elements can additionally or alternatively extend circumferentially around the package, e.g., in the shape of a ring or helix.

Moreover, relationships between elements can interfere with each others' packaging in various ways. One of these ways is that neither of two straight, stiff elements which have an expanded configuration putting them substantially at right angles to each other can be much longer than the diameter of the delivery configuration, unless that right angle is somehow made more oblique in the delivery configuration. The constraint is substantially absolute if one of two elements is constrained to extend parallel to a longitudinal axis of the packaged configuration. It may relax slightly if the two sides making the right angle can be somehow oriented to make an angle that points radially outward. This constraint can also arise even when one of the elements is shorter than the package diameter, e.g., due to space occupied by the other element, and/or occupied by yet other elements of the device. It can also happen for angles other than right angles, according to the general rule that the more obliquely two elements are angled with respect to each other, the more permissive are packaging-related constraints on length, other limitations being ignored.

In some embodiments of the present disclosure, a tissue anchor comprises an elongated element with a sharp distal end; wherein the element protrudes, in a deployed configuration, from a proximal-side supporting element that is itself elongated; and furthermore, protrudes at, or close to, a right angle with that supporting element. This angle should also be stable, but the need for stability potentially interferes with the introduction of flexibility to allow making it more oblique in the delivery configuration. Conversely, for a sufficiently elongated tissue anchor, there are potential difficulties in packaging the anchor element and the support element into a delivery configuration, without compromising anchoring functionality.

The feature of near-right-angle protrusion is preferable, in particular, when the movements which drive the tissue anchor into tissue are generated by a movement of the supporting element which is also at a near right-angle to its elongated axis. The anchor may insert to tissue more effectively (e.g., more reliably, deeper, and/or with reduced force requirements) when it points straight along the axis of its motion, rather than obliquely. Examples of supporting members that can move in this fashion include arm segments swiveling on a hinge (e.g., a hinge positioned distal to the arm segment's connection with of the tissue anchor), and cylindrical arrangements of struts expanding radially.

As to the anchoring element's elongation, retaining force is potentially increased if the anchor penetrates more deeply within tissue. However, full perforation of tissue can lead to undesired outcomes such as blood leakage. If the anchor is barbed, for example, the retaining reliability of the barb protrusion depends in part on the tissue which closes behind it having and retaining enough strength to resist tearing, even though it is penetrated. In some embodiments, the anchoring element is long enough to penetrate tissue without fully perforating it, e.g., to a depth of about 0.8-4 mm. In embodiments, for example, the anchors are about 2.6 mm long, optionally in a range of lengths between about 1.5 and about 3.4 mm.

However, an elongated anchor has a potential disadvantage impacting on its stability: the longer it is, the more levering advantage may act to deflect the anchor's sharpened point away from its preferred orientation along the axis of its motion.

Strengthening the joint of anchor and supporting member may reduce this disadvantage. However, as already mentioned, potential design problems then arise in the packaging of the device: stiffening the joint in an expanded configuration potentially interferes with providing an oblique angle in the delivery configuration. There is, for example, the issue of how much constraining force can be exerted during confinement in a delivery sheath, without problems such as package-sheath friction interference, and/or increased risk of catastrophic failure.

In some embodiments of the present invention, an anchoring element is provided as an insert that interlocks with surfaces of a receiving aperture of the supporting element (which may be, for example, an arm of an LAA closure clip, or another tissue anchoring device). Interlocking between the anchoring element and the receiving aperture is shaped to resist removal from the receiving aperture by forces it is likely to encounter during operation after deployment; for example, by incorporating clasping shapes that require deformation well beyond what can be exerted by soft tissue in order to deform enough to dislodge.

Moreover, the anchoring element is shaped, in some embodiments, so that it incorporates an elastic member (e.g., a bar-spring) which deforms to allow interconversion between a collapsed configuration and an expanded configuration. In an expanded configuration, an elongated member comprising a distal point of the anchoring element is oriented so that it extends at substantially a right angle (e.g., within 10° of a right angle) away from a longitudinal axis of the supporting element. In a collapsed orientation, the near right-angle becomes oblique, for example, obtuse, deviating from the angle of the expanded configuration by at least 45°, and optionally 60° or more. In some embodiments, in the collapsed configuration, a distal portion of the sharp distal end is positioned relatively close to a longitudinal axis of the arm (that is, a longitudinal axis extending through and along the arm in the region at which the anchoring element is attached to it), compared to its position in the expanded configuration.

In some embodiments, interconversion between the collapsed and the expanded orientations comprises a relaxation of spring forces stored in the elastic member. In some embodiments, the relaxation of spring forces is accompanied by configuration changes that make the anchor element more rigid. In effect, the anchor element is configured to be relatively compliant when collapsed, but relatively rigid once expanded. This conversion is accomplished through changing interactions among the different parts of the anchoring mechanism.

In some embodiments, an interlocking attachment between the anchoring element and the receiving aperture to which it connects is provided; and divided between a plurality of base regions. In some embodiments, one of the base regions is left free to rotate and/or slide where it contacts the receiving aperture as the elastic member deforms. However, the freedom to rotate can be limited by the shape of the interlocking attachment, so that attempts to over-rotate the elongated member with the sharpened tip (e.g., beyond a right angle or other selected angle) are substantially prevented. In effect this helps rigidify the connection between the elongated member with a sharpened point and the supporting member, at least in one direction, but only once the two members are in their deployed orientation with respect to each other.

It is further arranged, in some embodiments, that the elastic member is oriented in the expanded configuration so that it acts as a brace against forces exerted in at least one direction. In the case of a bar spring acting as the elastic member, for example, the bar spring has a long axis and a short axis. It is relatively resistant to deformation (e.g., incompressible or non-extending) when force is exerted along the long axis, compared to force exerted along the short axis, which easily deforms it. If it does deform in response to force exerted primarily along the long axis, the bar spring may do so by buckling (bending about its middle), which distributes forces of compression and tension differently than bending which occurs monotonically as compression or tension exerted along a given side of the bar spring.

The elastic member connects to an interlocking attachment with the supporting member on one side (its proximal side), and on the other (its distal side), attaches to the elongated member with the sharp tip at a position away from its own attachment to the supporting member, for example, at a position between about ⅓ and ⅔ of the way to the sharpened distal tip.

The elastic member extends away from this region of attachment at an angle of, for example, at least 30°; optionally 45° or more, and connects to its base. This in effect creates a brace for the elongated member with the sharp tip. A significant part of forces exerted to on the tip of the elongated member to rotate the elongated member further will be directed along the relatively inelastic dimension of the elastic member, and so be prevented from contributing to further elastic deformation. Thus, in some embodiments, the elastic member acting as a brace and the rotation-limited interlocking attachment of the elongated member act together to prevent over-rotation of the elongated member past its selected angle of protrusion from the support element.

As for torquing forces exerted in the opposite direction upon the sharpened tip of the elongated member, the elastic member has the effect of shortening the lever arm upon which those force can act. The region attachment being relatively loose, the fulcrum upon which the torquing forces act tends to move to the next least-resistant element; distally, away from the supporting element, and toward the elastic member. For example the fulcrum of movement may move to about a midpoint of the elastic member. This distance is shorter, in some embodiments, than the tip-to-base distance, so it potentially increases the amount of force needed to create bending, compared to embodiments where the base itself is the fulcrum.

Moreover, in some embodiments, when the tip is bent to the oblique angle as it is in the delivery configuration, the elastic member may be arranged so that the lever arm length increases. Accordingly, it may require significantly less force to maintain the anchor element in its collapsed delivery configuration than is needed to deflect it from its deployed expanded configuration. This is a potential advantage for keeping constraining forces low in the packaged state, while still preserving resistance to deformation when the device is eventually deployed.

It should be noted that the anchoring element, including its clasps, elastic member, elongated member, and sharpened tip may in some embodiments be designed for manufacture as single piece, e.g., a piece readily cut in a single piece from sheet stock material, for example, laser cut from nitinol sheet stock. The piece may be separate from the main body of the closure device. In some embodiments, the anchoring element is snap-fitted to its region of attachment to the closure device, e.g., squeezed and/or pressed into place, after which it may become difficult to dislodge without the exertion of considerable force.

In some embodiments, the elastic member in particular is manufactured as a cut-out portion of an arm of a main body of the closure device (e.g., a strip cut from stock sheet used to make the arm, but left attached on one side). The anchoring element with one or more clasps, elongated member, and sharpened tip may interlock with the elastic member (e.g., via one or more interlocking slots). This anchoring element variation may be manufactured, for example, by cutout from a piece of stock sheet (e.g., nitinol stock).

There are thus, in some embodiments, mechanisms acting to do one or both of (1) strongly resist over-rotation of the anchor element by blocking rotation past a selected angle, and (2) resist re-folding of the anchor element toward its collapsed delivery configuration, with the resistance to re-folding being resistant to movement by a torque higher than a torque needed to maintain the anchoring element in its previous delivery configuration.

### Multipart Anchor Construction

An aspect of some embodiments of the present disclosure relates to tissue anchors for tissue-implanted devices constructed from a plurality of separate and interlocking components.

There may be material constraints on the construction of near right-angle joins between the anchoring element and its supporting elements, particularly but not only if those joints are to be flexible.

In some embodiments, device components are created by cutting (e.g., laser cutting) blanks from stock sheets; for example, sheets having a thickness in the range of 150 μm-600 μm. It should be also recognized that the supporting members cut from the blank may be finely constructed (e.g., no more than 1-3 times wider than the material thickness being folded). While a superelastic material such as nitinol is potentially capable of huge conformational changes, it still has some structural limits on its forming. Working from the cut blank, creasing to introduce sharp right angle bends tends to create areas of stress focus (and weakness) in already delicately shaped member; if, indeed, the bend can be introduced at all without snapping the material. It should be understood that structural integrity is paramount in tissue-implanted medical devices, particularly devices implanted where a broken-off piece could end up travelling through the circulatory system.

In some embodiments, a minimum radius of curvature which is acceptable for introduction into the formed blank in the vicinity where anchor and supporting element meet is about 5 mm, 6 mm, or more (e.g., for a blank thickness in a range of 150 μm-600 μm). This minimum radius can potentially be reduced for thinner stock material; however, it is potentially difficult or undesirable to adjust blanks to a thickness other than the sheet stock thickness, due to issues with material machinability, and/or risk of introducing regions of stress focus and/or embrittling.

If, instead, a whole component is created out of a thinner stock material, this may negatively impact upon other features of the component. For example, an arm component of a clip (such as an LAA closure device) may need to have enough thickness to allow it to provide transmit force to achieve anchoring, and sustain the holding together of tissue.

Accordingly, there is a potential problem which arises in the single-piece construction (that is, construction from a single cut blank) of an anchor which leads off of an arm at approximately a right angle. Achieving the angle through a large radius of curvature means that there is a larger extent of material which can spring-deform during an attempt to anchor a device, potentially leading to failure of device implantation. On the other hand, a sharper right angle may be unachievable or unsafe, unless other design qualities are compromised. The problem is exacerbated by technical limitations which may arise in the manufacturing of small components, related to problems of handling, temperature control, mechanical vulnerability, and the like.

In some embodiments of the present disclosure, anchoring mechanisms are formed by mounting an anchoring element to a supporting element, wherein each of the elements is formed from a separate blank. In some embodiments, the anchoring element itself is shaped with clasping shapes which are used to grasp the supporting element, optionally suitably formed shapes cut into the supporting element, for example, apertures and/or notches. In some embodiments, one or more additional pieces is used for support and retaining.

The two (or more) piece assembly of anchors potentially allows the mixing of component properties with more freedom and/or ease of manufacture than forming from a single blank provides. For example, use of thinner blank material for the anchoring element may, by its thinness more easily penetrate tissue to anchor. Use of a tubular blank material (e.g., sharpened to a needle point) may allow increased stiffness while maintaining a relatively small size.

### Attachment and Release Mechanism

An aspect of some embodiments of the present disclosure relates to the releasable, and optionally re-attachable attachment of a suturing clip to its delivery system.

In some embodiments, the mechanism comprises an interference fit between shapes of arms of the delivery device, and corresponding receiving shapes on a core of the suturing clip. While locking member is advanced, its presses the arms into their receiving shapes, holding the suturing clip in place. Withdrawal of the locking member allows the arms in turn to exit the receiving shapes, releasing the core and the suturing clip. Optionally, the process is reversed, which potentially allows retrieval of a device, even after it has been fully deployed and released.

An aspect of reversing deployment after engagement with tissue is the use of an anchor length which, while long enough to support engagement in the first lace, remains short enough to detach from tissue safely (e.g., without tearing or otherwise leaving behind an open hole). In some embodiments of the present disclosure, the anchor length is in a range of 0.8 mm to 4 mm; for example, about 2.6 mm. In some embodiments, the tissue engaged and/or disengaged from has a thickness of less than 6 mm; for example, less than 5 mm, 4 mm, 3 mm, or 2 mm thick. It is a potential advantage in particular for an LAA closure device to engage with tissue of the LAA ostium, since the tissue there tends to be thicker than tissue deeper in. This may provide relatively higher stability of device fixation.

### Ablation

In some embodiments, the tissue closure device is used additionally or alternatively for delivery of ablation energy to the ostium of the LAA. This is optionally done as a treatment for heart conduction disorders such as atrial fibrillation, in order to electrically isolate possible electrogenic zones in the LAA from the rest of the heart. Surfaces of the device are optionally electrically isolated from blood contact (e.g, by a coating of paralene or another polymer), leaving the anchors or some portion thereof uninsulated to allow direct contact with tissue to be ablated. Radiofrequency (RF) or electroporation energy (for example) is optionally delivered via electrical connection over the delivery device.

In some embodiments, anchors of the closure device are deployed enough to engage with tissue around the LAA and/or its ostium. Electrical energy is delivered through the anchors to the tissue, ablating it so that electrical impulse transmission across the resulting scar is prevented.

The ablation may be performed one or more than one time; for example, first when the tissue is engaged but the closure device remains uncollapsed, and then again after full collapse of the closure device.

Additionally or alternatively, reversing engagement may be performed, in some embodiments, in conjunction with use of the device for delivering ablation energy to the ostium of the LAA. In some embodiments, a first engagement of anchors is performed at a first rotational angle, and RF energy delivered through the device and its anchors into tissue. The device is then disengaged, rotated and re-engaged. Optionally, ablation is performed again at the new engagement location, which potentially helps ensure overlap of scars.

Moreover, since deployment of the device is reversible: in some embodiments the device is not used for closure, but only for ablation. Anchors of a device are optionally specially configured for this purpose; for example, the device is provided with anchors of a design which is more readily reversible in its engagement (e.g., is not barbed), and/or with contacts which press against tissue without penetrating it. Such devices are optionally sized and shaped with electrodes that position upon deployment to press against an orifice of a tissue target other than the LAA, for example, shaped to press against the lumen of a pulmonary vein and/or pulmonary vein ostium, where it may be used, in some embodiments, to perform pulmonary vein isolation treatment.

### Positioning

An aspect of some embodiments of the present disclosure relates to positioning of a deployment system in preparation to deploy a suturing clip to close an LAA.

In some embodiments, proper pre-deployment positioning is achieved by focusing on the control of three degrees of freedom: a correct predetermined rotational orientation of the suturing clip, centering of the delivery system relative to the LAA ostium along the anterior/posterior axis, and advancing of the delivery system by about 1-8 mm past the circumflex coronary artery.

In some embodiments, these degrees of freedom are addressed in sequence, e.g., in the sequence just listed. In some embodiments, setting of each degree of freedom is performed under imaging observation, optionally using different view angles and/or imaging modalities, as appropriate.

For example, in some embodiments:
The orientation of the suturing clip is set under fluoroscopic visualization;
Anterior/posterior centering of the delivery system is set using ultrasound imaging, optionally 3-D imaging; and
Pre-deployment insertion depth of the delivery system is set using planar ultrasound imaging that includes a plane of the circumflex coronary artery.

In some embodiments, a distal end of an overtube of the deployment system is provided with elements configured to enhance visualization under both ultrasound and fluoroscopic observation. In some embodiments, an ultrasound enhancement element comprises a braided sleeve. Optionally, a metal cylindrical capsule which contains the suturing clip is provided with a roughened surface to enhance its echogenicity. In some embodiments, a radiopaque marker is embedded in the distal end of the overtube, for example, a ring. The ring comprises, for example, tungsten, tantalum, gold, or another metal. In some embodiments, the distal end of the overtube comprises both a capsule made of a first metal, and a radiopaque marker of a material having a greater radiopacity than the first metal. Optionally, the first metal comprises stainless steel and/or nitinol. Optionally, the second metal comprises tungsten, tantalum, and/or gold.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Expandable/Collapsible Suturing Clips

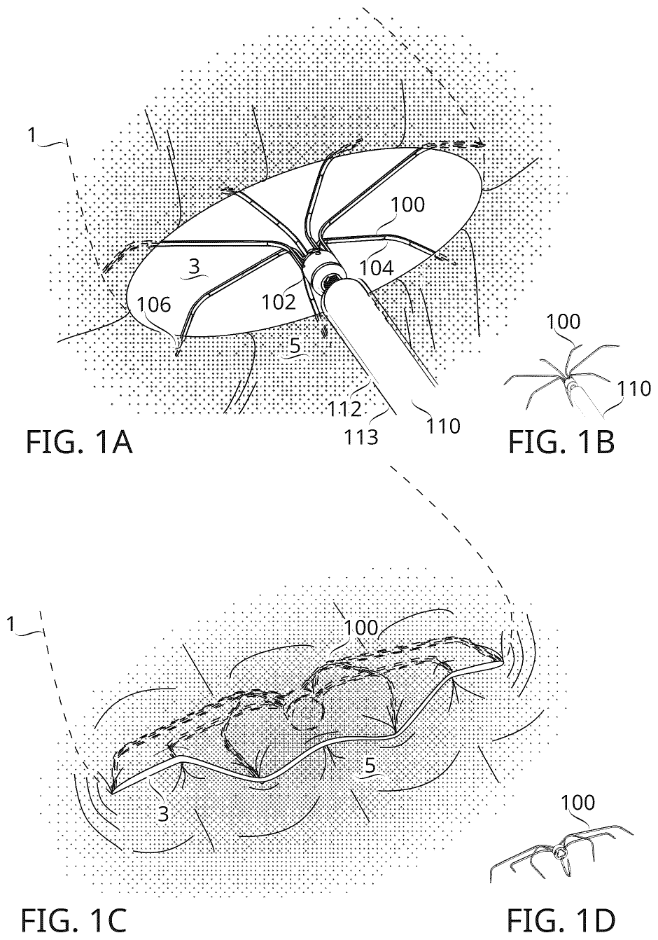

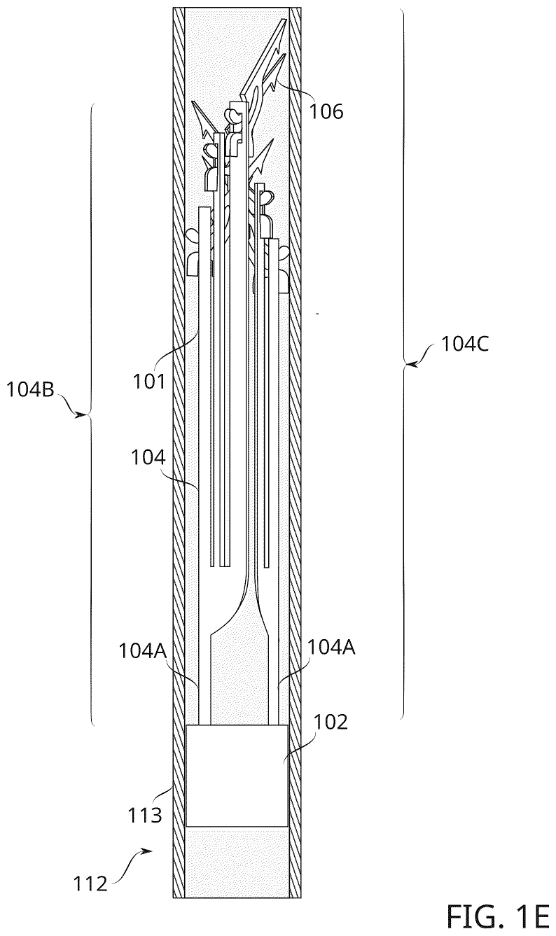

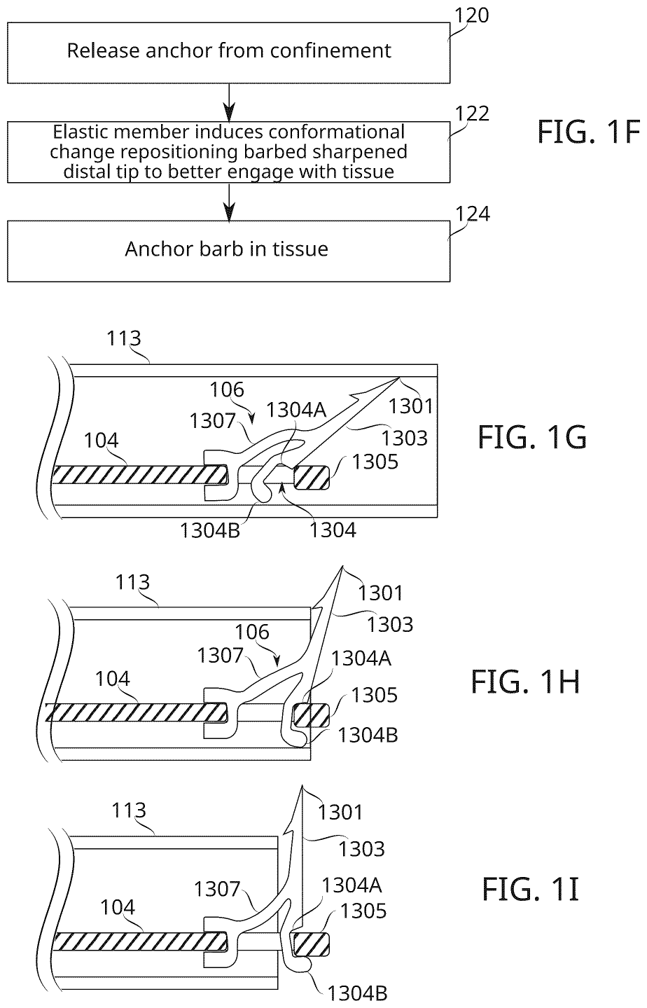

Reference is now made to FIG. 1A, which schematically illustrates a suturing clip 100 engaged to tissue within a LAA 1, according to some embodiments of the present disclosure. Reference is also made to FIG. 1B, which schematically illustrates the suturing clip 100 of FIG. 1A in an expanded configuration suitable for tissue engagement, according to some embodiments of the present disclosure. Further reference is made to FIG. 1C, which schematically illustrates a suturing clip 100 which has engaged tissue within an LAA 1 and then undergone a conformational change to close an LAA ostium 3, according to some embodiments of the present disclosure. Reference is also made to FIG. 1D, which schematically illustrates the suturing clip 100 of in the LAA-closing conformation of FIG. 1C, according to some embodiments of the present disclosure. Reference is further made to FIG. 1E, which schematically illustrates a suturing clip 101 confined to a delivery configuration by an overtube 113.

Before expansion to the expanded configuration of FIGS. 1A-1B, the suturing clip 100 is confined, in some embodiments within an overtube 113 of a distal end 112 of a catheter or other delivery system 110, for example as described in relation to FIG. 1E. The suturing clips shown in FIGS. 1A-1D are representative of two expanded configurations of the suturing clip: a partially deployed configuration in FIGS. 1A-1B, and fully deployed configuration in FIGS. 1C-1D.

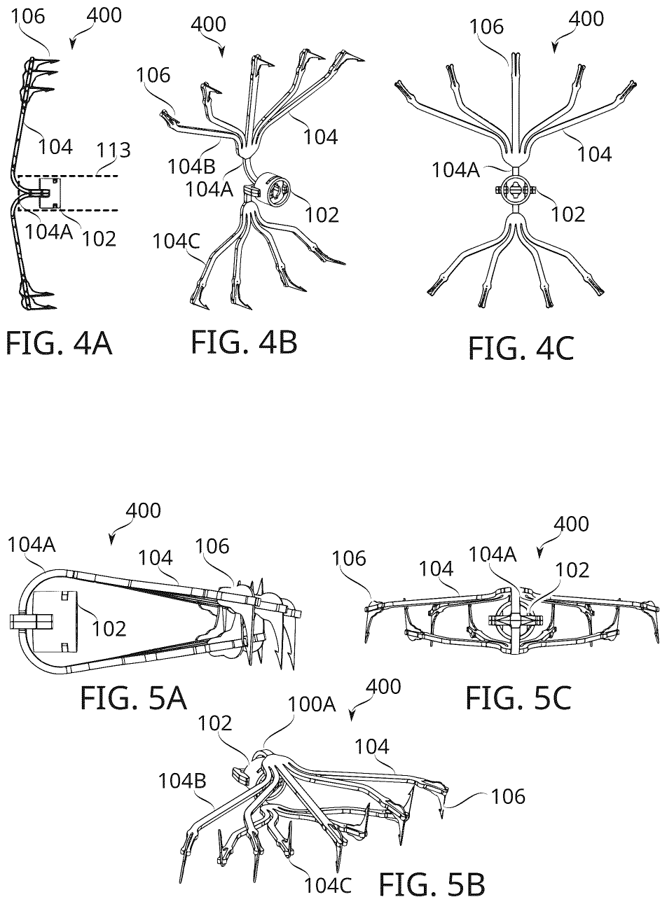

Suturing clip 100 is optionally configured according to any of a range of forms and designs, more particular examples and/or details of which include suturing clips described in relation to FIG. 4A-12B herein, and/or as described in International Patent Publication No. WO2019/207585, the contents of which are included herein by reference in their entirety. Embodiments may vary from one another, for example, in numbers of arms, specifics of arm shape, specifics of anchor shape, specifics of core design, and type of closure. Additionally or alternatively, embodiments of suturing clip 100 encompass any suitable arrangement of anchoring positions. For example, in the expanded anchoring configuration, the anchors are optionally arranged in a circular, elliptical, oval, split-curve (arranged along two or more perimeter sections), split-line (arranged along two or more line segments), and/or open-sided perimeter configuration. In the collapsed suturing configuration, for example, the anchors are optionally arranged in a circular (e.g., collapsed toward a common center), linear, zigzag, or another aperture closing configuration. It should be noted that the “collapsed suturing configuration” is considered herein to be one of the expanded (deployed) configurations of suturing clip 100, considered relative to the collapsed delivery configuration that precedes its expansion.

Generic features of a suturing clip 100 introduced in the descriptions of FIGS. 1A-1E should be understood to apply to any of the more particular embodiments herein, modified as explained in their more particular descriptions.

FIGS. 1A and 1C are drawn from a perspective on an atrial side of an LAA, looking at the LAA ostium 3, which is an opening in atrial wall 5 leading into LAA 1. Figures IA and IC show selected phases in the closure of LAA 1 using suturing clip 100.

FIG. 1A shows suturing clip 100 expanded within LAA 1, and FIG. 1B shows the same expanded suturing clip 100 without illustrating the surrounding tissue. Arms 104 of suturing clip 100 each deploy radially from their respective bases, where they connect to core 102. Anchors 106, positioned on respective terminal ends (i.e., ends opposite the ends attached to core 102; also referred to herein as “free ends”) of arms 100, are configured to engage tissue from within LAA 1, for example by piercing, hooking, and/or pinching. In FIG. 1A, core 102 of suture clip 100 is connected to delivery system 110. Delivery system 110 comprises, in some embodiments, a catheter device, for example a percutaneous catheter device, of which distal catheter end 112 is illustrated. Previous to achieving the expanded position of FIG. 1A, suturing clip 100 was delivered by delivery system 110, e.g., enclosed within a lumen of distal catheter end 112 in a collapsed delivery configuration, for example as described in relation to FIG. 1E.

In some embodiments, arms 104 are electrically insulated metal, while some portion of anchors 106 is left bare and capable of making good electrical contact with tissue. Coupled to an electrical energy (e.g., RF energy and/or electroporation energy) delivery conduit (e.g., via delivery system 110), suturing clip 100 is thereby configured to act additionally or alternatively as a tissue ablation device. Operated in this mode to deliver electrical energy, the device induces scarring at multiple locations simultaneously around a circumference of the LAA ostium 3. This potentially creates a block that prevents ectopically originating electrical impulses from crossing from the LAA into the rest of the heart. Ablation is optionally performed at the configuration shown in FIG. 1A, optionally again after a rotation of the device to place the anchors in positions between their previous positions, and/or optionally while the device is in the collapsed position shown in FIG. 1C (before disengagement with delivery system 110). Ablations in different positions of engagement and/or states of collapse potentially help to ensure completeness of block.

FIG. 1C shows suturing clip 100 in a collapsed suturing configuration, still engaged with tissue of LAA 1, and FIG. 1D shows the same collapsed suturing configuration of suturing clip 100 without illustrating the surrounding tissue. In the collapsed suturing configuration, suturing clip 100 draws the LAA ostium 3 closed from its position within LAA, creating a band of closure (optionally a zigzag band of closure as illustrated). LAA ostium 3 is closed potentially without any exposure of suturing clip 100 along an atrial side of atrial wall 5. For example, suturing clip 100 is entirely within the LAA 1 on the side of the closed LAA ostium 3, and/or suturing clip 100 is unexposed to the lumenal contents (e.g., blood) on the atrial side of atrial wall 5. This is a potential advantage for preventing device-induced thrombogenesis producing blood clots having access to the circulating blood pool. Attachment at the ostium 3 and/or adjacent to the ostium 3 is potentially advantageous over attachment at other positions within the LAA, insofar as tissue there may be thicker and/or stronger than tissue within the inner LAA.

In FIG. 1E, a suture clip 101 exemplifying an alternative design of a suture clip 100 is shown, this time in a collapsed delivery configuration, confined within overtube 113. Core 102 is positioned on a proximal side of suture clip 101, with arm assemblies 104B, 104C extending distally core 102. The arm assemblies 104B, 104C are configured as branched groups of arms 104, each group connected to core 102 through a common strut 104A. Also shown are anchors 106. In the example shown, anchors 106 correspond to the anchor design of FIGS. 7A-8B, however, another anchor configuration may be provided additionally or alternatively to the configuration shown. In the example of FIG. 1E, there are five arms 104 branching from strut 104A of arm assembly 104B, and four arms 104 branching from strut 104A of arm assembly 104B; some arms are hidden in the view of FIG. 1E behind symmetrical counterparts. Various expanded views of a version of this arm arrangement are described, for example, in relation to FIGS. 4A-5C, herein.

### Clip Configuration Changes During LAA Closure

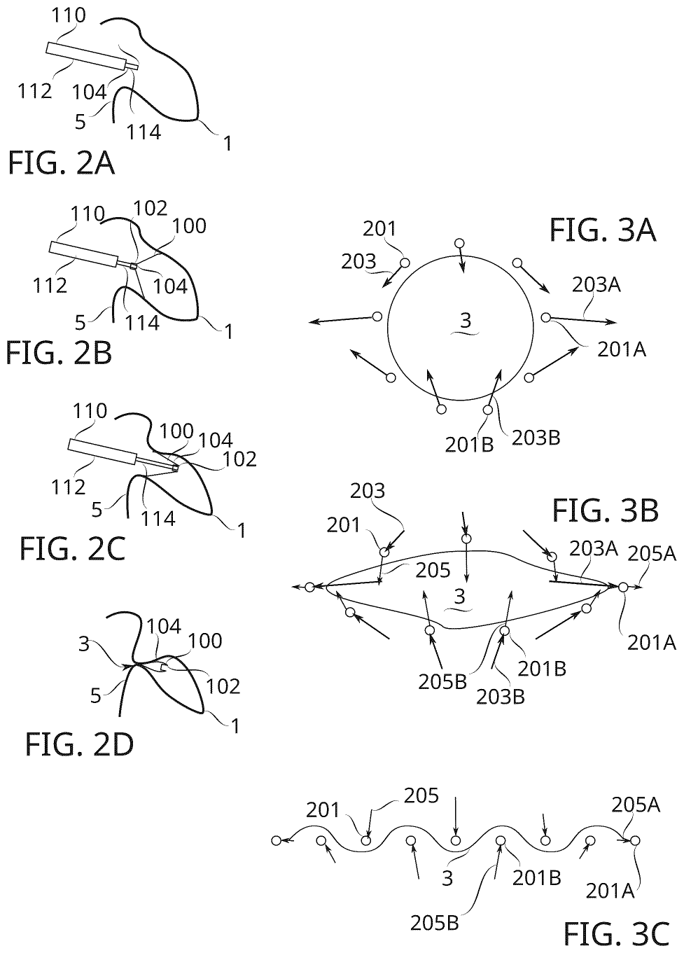

Reference is now made to FIGS. 2A-2D, which schematically illustrate stages in the deployment of a suturing clip 100, according to some embodiments of the present disclosure. Herein, the reference characters “100” are used in descriptions of suturing clips in general, with certain more particular embodiments being given their own reference characters identifying them as species of the genus, and/or described with reference to their particular characteristics. Reference is also made to FIG. 1F, which is a schematic flowchart of operations related to an anchor 106 of a suturing clip 100, according to some embodiments of the present disclosure. Further reference is now made to FIGS. 1G-1I, which together schematically illustrate a method of expanding, and optionally re-collapsing, an anchor of a suture clip, according to some embodiments of the present disclosure.

FIG. 2A, in some embodiments, represents the approach of a delivery system 110 including a distal catheter end 112 toward an ostium 3 of a left atrial appendage 1 in the atrial wall 5 of a heart atrium, and the partial unsheathing of a suture clip 100 thereat.

In some embodiments, partial unsheathing of suturing clip 100 (either at an early stage corresponding to FIG. 2A or a later stage of unsheathing) also releases anchors 106 from confinement, allowing them to undergo a conformational changes as part of their deployment. Examples of anchors 106 which undergo a conformational change upon deployment are described, for example, in relation to FIGS. 1G-1I, 6A-8B and 12A-12B. At block 120 of FIG. 1F, in some embodiments, anchor 106 is released from confinement, e.g, by partial unsheathing of a portion of suture clip 100 which carries anchor 106. At block 122, in some embodiments, the anchor 106 undergoes a conformational change. In some embodiments, the conformational change causes a sharpened distal tip of the anchor 106 to move away from a body of the arm that carries it, e.g., away from a base of the anchor 106 which is attached to the arm 104. The conformational optionally comprises a bending motion along a body of anchor 106, which erects the tip to re-orient to an angle perpendicular or more nearly perpendicular to the arm 104 at the site of anchor 106 attachment. In some embodiments, the conformational change is driven by energy stored by the deformation of the anchor 106 when it is packaged (and confined) previous to introduction into the body. In some embodiments, anchor 106 comprises a superelastic material (e.g., nitinol). The conformational change of the anchor 106 repositions (e.g., re-orients) the sharpened distal tip of anchor 106, potentially assisting its anchoring into tissue, for example as further described in relation to block 124 of FIG. 1F and in relation to FIG. 2B, hereinbelow.

As an example, FIG. 1G depicts an anchor element 106 of a suture clip or other implantable device, mounted to a mounting element which in some embodiments comprises a portion of an arm 104 of the suture clip. Arm 104 is shown in cross section to allow details of the movement of anchor element 106 to be shown. Anchor element 106 is confined within an overtube 113 or other delivery packaging, and held in a collapsed state by interference from walls of the delivery packaging; e.g., by force exerted between overtube 113 and anchor tip 1301 on a distal side of anchor element 106, and overtube 113 and lower member 1304B on a proximal (attached) side of anchor element 106. The confinement is resisted by spring force stored in elastic member 1307, and forces elongated member 1303 (which terminates in tip 1301) to an oblique angle with respect to its mounting arm 104. Furthermore, the confinement prevents recess 1304 from engaging with distal end 1305 of mounting arm 104. In some embodiments, the spring forces are uniform in polarity along the sides of the elastic member 1307, that is, a side of elastic member 1307 is in tension along its full length, and another side is in compression along its full length.

In FIG. 1H, overtube 113 has been partially removed from around anchor element 106 (e.g., by withdrawing the overtube 113 and/or advancing anchor element 106), allowing anchor element 106 to begin expansion. The expansion comprises a sliding movement of elongated element 1303 with a long distal end 1305, with the result that recess 104 is brought into alignment with distal end 1305. Upon alignment, an upper edge 1304A and a lower member 1304B which define upper and lower sides of recess 1304 advance in an arm-distal direction (rightward, in the figure) under the force of tension exerted via elastic member 1307. As a result, a portion of distal end 1305 is positioned between upper edge 1304A and lower member 1304B.

In FIG. 1I, overtube has been sufficiently removed from around anchor element 106 that it is free to fully expand. In the further expansion movements of anchor element 106, recess 1304 and distal end 1305 form a pivot. More particularly, in some embodiments, the fulcrum of the further angular movements of elongated member 103 is confined to a region of contact located within recess 1304; that is, located upon and/or between upper edge 1304A and lower member 1304B.

From the fully expanded configuration of anchor element 106, in some embodiments, further deformation of elastic member 1307—if enough force is exerted—comprises a buckling mode of deformation, with the ends of one side of elastic member 1307 in tension and the middle of that side in compression; and on another side, the ends in compression and the middle in tension.

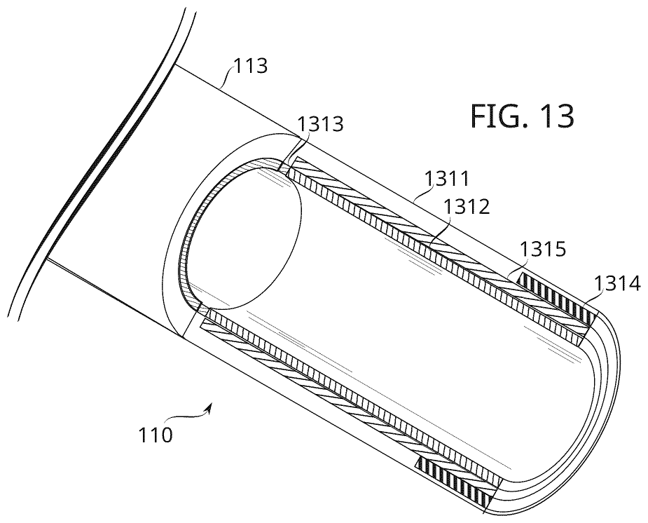

In some embodiments, an anchor element 106 in an expanded condition (as in FIG. 1G, for example) can optionally be withdrawn again into overtube 113, e.g., via the stages illustrated in FIGS. 1H and 1G. Optionally, a larger sleeve or overtube is introduced over overtube 113, potentially increasing the leverage available to assist in collapsing anchor elements 106. Optionally, the distal tip of overtube 113 comprises an inner metallic cylindrical capsule (e.g., a layer 1302 as described in relation to FIG. 13), strong enough to resist damage such as cutting or puncture as anchor element 106 is withdrawn into and/or through it. The metal capsule may provide ultrasound echogenicity to the tip; in some embodiments, the metal capsule is provided with ridges, grooves, slots, and/or another structure to enhance echogenicity.

Re-collapse may be done, for example, to increase safety during repositioning of a partially deployed device, and or because there is a reason to abort device implantation, or retrieve the device in a subsequent procedure. During withdrawal, there may be a phase of higher initial resistance to collapse before recess 104 is disengaged (e.g., during the “pivoting” stage of expansion), followed by a phase of lower resistance to collapse once recess 104 is disengaged, wherein additional deformation comprises sliding motion along a more anchor-distal surface of elongated member 1303. This at least biphasic sequence of forces has a potential advantage for conferring increased resistance to collapse when the anchor element 106 is in its deployed, operational state, but allowing the anchor element 106 to press against its confinement with decreased restorative force when it is fully packaged for delivery.

FIG. 2B, in some embodiments, represents further unsheathing of suturing clip 100 by actuation of delivery system 110. Unsheathing comprises, for example, retraction proximally of distal catheter end 112 from delivery mount 114, and/or extrusion distally of delivery mount 114 from catheter end 112. In the example of FIG. 2B, suturing clip 100 is packaged with core 102 on a distal side of the collapsed delivery configuration, and anchoring ends of arms 104 on a proximal side of the collapsed delivery configuration. The reverse packaging configuration (with arms 104 on the proximal side of core 102) is also an option provided in some embodiments, for example, embodiments as described in relation to International Patent Publication No. WO2019/207585.

Arms 104 of suturing clip 100 are expanded radially away from core 102 (relative to the position of FIG. 2A), and they make an initial engagement (via anchors 106, not illustrated) with a lumenal side of tissue within LAA 1. In some embodiments, anchors 106 are at this point oriented pointing distally, while also being placed where they press outward against tissue they contact (and also somewhat point into it). Accordingly (e.g., as an example of block 124 of FIG. 1F), a backward tug on suturing clip 100 may act to sink the anchors into the tissue they contact, for example, tissue of LAA 1, and/or of an ostium 3 of the LAA. In some embodiments, anchor 106 is barbed (e.g., narrows in width proximal to a wider distal part of the anchor). Once the barb sinks into tissue (e.g., as in block 126 of FIG. 1F), the force needed to remove the barb from tissue is potentially somewhat increased, compared to an unbarred point. It should be noted that deliberate release from tissue in particular (e.g., release accomplished by pushing the device distally again) is not necessarily prevented by the barb, particularly right away after penetration when the entrance cut is fresh. After a period of healing, tissue may close superficial to the wider distal part of the barb, potentially enhancing resistance to unintended barb release.

It may also be noted that the tenacity of securing by the barbs potentially passes through a plurality of states as suturing clip 100 is deployed. Upon initial penetration of tissue while in a partially deployed state (for example, as shown in FIG. 1A), barbed anchor tips may resist removal more strongly than a similar but un-barbed anchor tip would, but still be removable by exertion of an amount of force which can reasonably be exerted through the deployment mechanism. While undergoing full deployment (e.g., to the state shown in FIG. 1C), tissue may deform so that pulling straight out perpendicular to the tissue surface no longer urges the anchor's barb to move parallel along the passage it cut upon initial penetration. Moreover, anchors may be re-oriented relative to each other so that there is no common direction along which force can be exerted to remove them all.

Expansion of arms 104, in some embodiments, comprises a superelastic configuration change due to arms 104 being set, when unconstrained, to expand as shown. Additionally or alternatively, a separately actuated mechanism forces expansion of arms 104.

FIG. 2C, in some embodiments, represents an intermediate stage of closure of arms 104 toward a collapsed suturing configuration. Different actuation mechanisms and/or shape transitions may be used to complete the closure of ostium 3.

Optionally, closure comprises a continued conformational change of arms 104, for example as shown in FIG. 2C. Herein, this type of closure is also referred to as an “everting” closure, wherein initially distally-pointing arms 104 (e.g., oriented as in FIG. 2A) continue to rotate past their most-expanded state, reversing their proximal-distal orientation relative to core 102 to point proximally. The suturing clips 100, 101 of FIGS. 1A-1E also represent “everting”-type suturing clips. A potential advantage of everting-type closure is a lowered distance of penetration into the LAA during insertion, reducing one or more of a risk of perforation and a risk of dislodging a pre-existing thrombus. In some embodiments, resilient elasticity of arms 104 is used to provide force for closure. Optionally another mechanism is provided, for example a sliding disc mechanism, e.g., as described in International Patent Publication No. WO2019/207585.

FIGS. 2D, in some embodiments, represents a final stage of closure, wherein the arms 104 have reached a fully closed collapsed suturing configuration. LAA ostium 3 is now closed. Delivery system 110 is detached, and suturing clip 100 remains attached to lumenal-side tissue of LAA 1, and sequestered within LAA. Insofar as suturing clip 100 is so-sequestered, it is prevented from acting as a center of thrombogenesis for blood clots which could enter the general circulation. Potentially, the sutured sides of the ostium 3 of LAA 1 will gradually grow together, preventing re-opening of LAA 1.

### Anchor Movements During LAA Closure

Reference is now made to FIGS. 3A-3C, which schematically illustrate movements 203, 203A, 203B, 205, 205A, 205B of anchoring positions 201, 201A, 201B over the course of a transition of a suturing clip 100 between a deployed-and-anchored configuration, and a collapsed suturing configuration, according to some embodiments of the present disclosure. The movements are shown as they would be viewed from a direction looking into the LAA through the aperture of ostium 3.

In FIG. 3A, anchoring positions 201 are initially arranged around an interior circumference of ostium 3. Herein the reference characters 201 refer generically to anchoring positions (of which one particular instance is labeled), and the reference characters for anchoring positions 201A, 201B refer to particularly labeled instances of an anchoring position 201. In the example illustrated, there are nine anchoring positions 201, for example, corresponding to the nine arms of the embodiment of FIGS. 4A-5C. Optionally, more or fewer anchoring positions (e.g., 5, 6, 7, 8, 10, 11, or 12 anchoring positions) are provided.

As a suturing clip 100 collapses towards its collapsed suturing configuration (FIG. 3B), the anchoring ends of each arm 104 move in different directions within the viewing plane. Arrows 203, 205 (as identified generically) each point in a different direction, indicating the position to which each anchoring position 201 moves between the configurations of FIG. 3A and the positions of FIG. 3B and then FIG. 3C.

It is noted in particular that there is motion of some anchoring positions (e.g., anchoring position 201A in directions 203A and then 205A) radially outward, while other anchoring positions (e.g., anchoring position 201B in directions 203B and then 205B) move generally radially inward. There is, however, a convergence in common to a shared suturing line. As a result, suturing clip 100 gradually draws ostium 3 into a new, nearly linear shape which retains most of the perimeter length of original ostium 3 (e.g., at least 50%, 70%, 80%, 90%, or 100% of the original perimeter length). Herein, the term “laterally-displacing collapse” is used to refer to collapsing movements of anchoring positions 201, arms 104, anchors 106, and/or other elements of a suturing clip 100, which act to stretch an aperture in one direction (i.e., laterally) while collapsing it in another.

FIG. 3C shows the anchoring positions 201 in the collapsed suturing configuration. The actual collapsed suturing configuration depends on a balance between forces placed on anchoring positions 201 urge them toward the same line—or even toward positions past the same line—and forces that resist this motion. In some embodiments, the result is a wavy (“clam shell”) or other irregular pattern along the lip of ostium 3. For example, tissue near to an anchoring position 201 tends to press further inward, while tissue further away from an anchoring position tends to give way to this pressure. To this extent, mechanical properties of the tissue itself become part of the seal created by the suturing clip 100 at the site of the ostium 3. In the collapsed suturing configuration, for example, the anchors 106 are optionally arranged in any suitable configuration for closure, e.g., a circular (e.g., collapsed toward a common center), linear, zigzag, or another aperture-closing configuration.

Reference is now made to FIGS. 4A-4C, which schematically represent views from different directions of a suturing clip 400 in a partially deployed and fully arm-expanded configuration, according to some embodiments of the present disclosure. Reference is also made to FIGS. 5A-5C, which schematically represent view from different directions of suturing clip 400 in a fully deployed collapsed suturing configuration, according to some embodiments of the present disclosure.

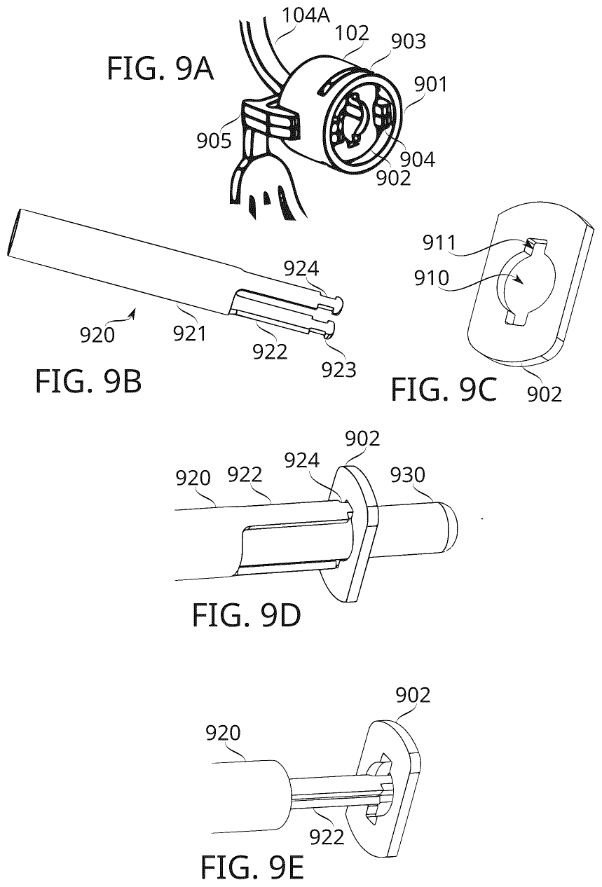

FIGS. 4A, 5A show suturing clip 400 in a side view (i.e., a perspective on the clip similar to the perspective of FIGS. 1E and 2A-2D), with proximal side on the right, and distal side on the left. The dotted lines in FIG. 4A represent the position of a confining overtube 113 relative to suturing clip 400, such that suturing clip 400 remains partially constrained in its movements, and thus is held with its arms splayed out to about their maximal extent. The configuration of FIGS. 5A-5C is reached upon complete removal over overtube 113, and subsequent detachment of suturing clip from its delivery system 110, for example as described in relation to FIGS. 9A-9E, herein.

FIGS. 4C, 5C show suturing clip 400 in a face on view from a proximal side (i.e., a perspective on the clip similar to the perspective of FIGS. 3A-3C).

FIGS. 4B, 5B show suturing clip 400 in perspective views, with the proximal side of the clip oriented toward the right.

In FIGS. 4A-5C, features including core 102, common strut 104A, arms 104, and anchors 106 described, e.g., in relation to FIG. 1E are illustrated in partially and fully deployed configurations. Arms 104 are biased during manufacture to splay out upon release from overtube 113. In the configuration shown in FIGS. 4A-4C, anchors 106 are oriented to point backward. This is optionally the configured used to establish tissue anchoring. The expanded arms hold the aperture (e.g., of the LAA 1) taut. A tug on the delivery system 100 can then be used to pull the sharp points of anchors 106 into tissue, before closure proceeds to completion. In the configuration shown in FIGS. 5A-5C, closure has been completed by collapse of suture clip 400 to its collapsed suturing configuration. It may be noted that arm assemblies 104B, 104C have interdigitated in FIG. 5B, which in some embodiments produces a “clamshell” closure as described, e.g., in relation to FIG. 3C.

### Expandable/Collapsible Anchoring Mechanisms

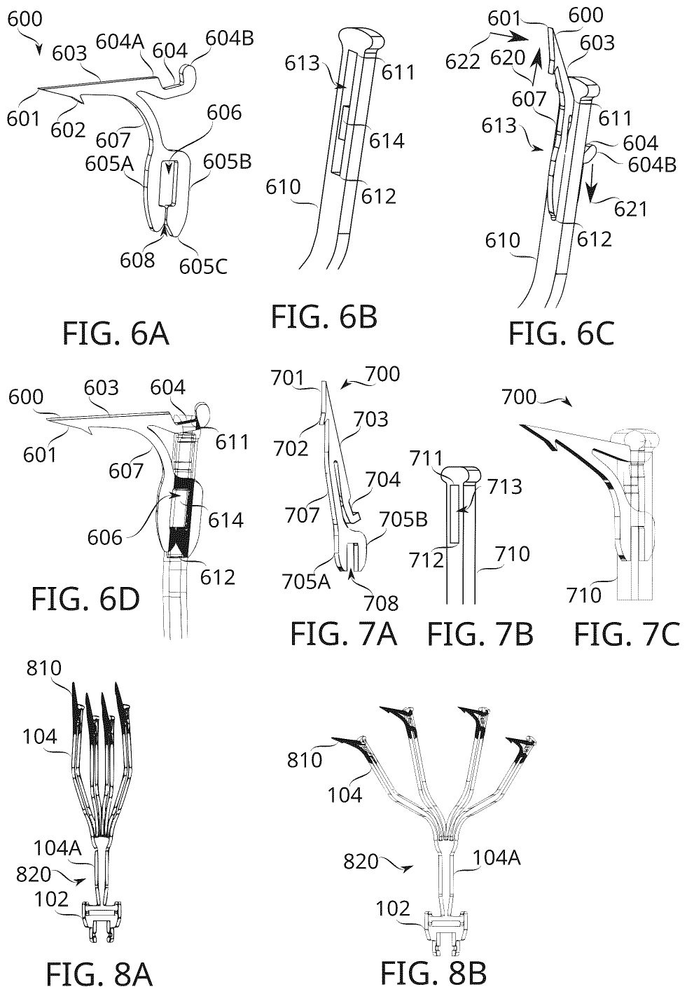

Reference is now made to FIGS. 6A-6D, which schematically represent a sprung tissue anchoring mechanism, according to some embodiments of the present disclosure.

In some embodiments, the sprung tissue anchoring mechanism is used to anchor an arm 104 of an LAA closure device 100 to tissue of the LAA. In FIGS. 6A-6D, distal arm section 610 is, for example, a distal part of an arm 104.

FIG. 6A illustrates an anchoring element 600. Anchoring itself is accomplished by elongated member 603, tipped with a sharpened tip 601 at a distal end of elongated member 603. In some embodiments, a barb 602 is provided which comprises a gradual widening from tip 601 in a proximal direction along elongated member 603, followed by a relatively sharp decrease in thickness. Optionally, an edge between the tip of barb 602 and a base of barb 602 where the edge connects with a shaft of elongated member 603 is refused so acutely as to place the base of the barb 602 at a position more distal than the wide point of the barb 602. The barb, in some embodiments, helps with anchoring by easing penetration of tissue as it move distally, generating leverage along its more gradual distal slope. Tissue can close in around the proximal side of the barb 602 after it passes, and may afterward help resist forces pulling the barb 602 proximally.

Anchoring element 600 has two regions of basal connection to distal arm section 610 (FIG. 6B), comprising recess 604 and recess 606, respectively. Recess 604 directly supports elongated member 603, and is defined between lower member 604B, and upper edge 604A. Recess 604 inserts, in some embodiments, into an arm-distal side of receiving aperture 613. When inserted, recess 604 reaches through receiving aperture 613 and slightly beyond it in the arm-distal direction to clasp distal wall 611 on either side of receiving aperture 613. The clasping is loose enough to permit swiveling of recess 604 with respect to distal wall 611. In some embodiments, recess 604 may respond to forces in one direction by sliding out of receiving aperture 613 altogether. As illustrated, recess 604 has an open-sided “C” shape. The elements 604A, 604B defining recess 604 are optionally extended to create a more closed shape, for example as next described in relation to recess 606.

Recess 606 is defined by upper member 605A, and lower member 605B, which, in some embodiments, branch from a common base near elastic member 607 to meet on one side of aperture 606 with a slight gap 608. Recess 606, in some embodiments, is sized to fit over a bar 614 within receiving aperture 613. The fit may be accomplished during manufacture by slightly distorting members 605A, 605B to open up gap 608 enough to slide over bar 614. In some embodiments, the fit between bar 614 and recess 606 is such as to resist rotation: for example, bar 614 is provided with a polygonal cross section (e.g, of 3 or 4 sides) which the shape of recess 606 duplicates. Additionally or alternatively, in some embodiments, one or more extensions of members 605A, 605B (e.g., extension 605C) extend proximally to interfere with proximal wall 612 aperture and resist rotation around bar 614.

Accordingly, when both recess 606 and recess 604 are engaged with their respective elements of receiving aperture 613, there is a two sided mounting provided (e.g., as shown in FIG. 6D). Recess 606 may provide most of the immobilizing strength of the connection. Recess 604 helps to stabilize the connection, but is free to pivot through a limited range of motion. In some embodiments, recess 604 may be partially free to slide as well.

Elastic member 607, in some embodiments, interconnects the structures defining recesses 606 and 604, including interconnection of elongated member 603 with the structures defining recess 606. Elastic member 607, in some embodiments, comprises a bar-spring, which is optionally formed from a single piece (e.g., cut from sheet stock), along with the other structures of anchoring element 600; e.g, those just described. In some embodiments, without external tension, elastic member 607 bends to a shape approximately as shown in FIG. 6A. Insertion into aperture 613 may generate some compression (by forcing recesses 604 and 606 closer to each other), but still leaves elongated member 603 pointing substantially in a direction orthogonal to arm section 610 and/or receiving aperture 613.

The orientation of elongated member 603 (e.g., as measured from tip 601 to a point of contact of recess 604 with arm section 610) need not be strictly orthogonal to arm section 610. A more nearly right-angled orientation may tend to better resist collapse as tip 601 is forced into tissue since, e.g., a smaller vector of that force is exerted along the distal-to-proximal axis of arm section 610 (at least, if anchoring force is also exerted along the longitudinal axis of elongated member 603).

Additionally or alternatively, the choice of protrusion angle may be adjusted according to the orientation arm section 610 assumes in some partially deployed configuration, such as is illustrated in FIGS. 5A-5C. During deployment, anchoring may be achieved by tugging proximally on a connection made with core 102, with anchors (e.g., anchors 106 of FIGS. 4A-4C) potentially entering tissue most easily if they encounter it at an orientation which is at a right angle to the tissue surface, but also parallel to the direction of force. However, the direction of force may not always be at a strict right angle to tissue surfaces. Optionally a compromise angle of made by anchor 106 with its mounting location on arm 104 is selected to be more oblique than a right angle, and/or arm 104 itself is oriented to point anchor 106 in a direction which is not parallel to a direction of movement (e.g, tugging) exerted to embed anchors 106 in tissue.

In some embodiments, elastic member 607 is integrated into distal arm section 610. It may be manufactured, for example as a protrusion into the cutout region of receiving aperture 613, a principle which is illustrated, for example, as barb 1104 of FIG. 11B. The elastic member 607 (of the arm itself) may be treated so that its relaxed position is bent out of receiving aperture 613. A recess shaped to have the clasping function of recess 606 (substantially wrap-around, or optionally open-sided, for example as shown for recess 708 of FIG. 7A) is optionally shaped at the free end of this variant of elastic member 607, and configure do clasp an anchoring element 600 without elastic member 607, for example, at a position along elongated member 603. Additionally or alternatively, a recess 606 is provided directly on elongated member 603 to clasp or otherwise engage with an elastic member 607 which is directly attached to the arm.

FIG. 6C shows anchoring element 600 inserted to receiving aperture 613, in a shape it may assume, in some embodiments, if a sufficient arm-distal directed force (e.g., in a direction indicated by arrow 620) is exerted to bend elastic member 607. As a result of the exerted force, elongated member 603 rotates against its contact with distal wall 611 so that tip 601 moves in the arm-distal direction. Recess 604 is pivoted in the opposite direction. Optionally, the pivoting disengages upper edge 604A of recess 604 from wall 611 allowing it to be pressed partially or fully through receiving aperture 613.

In some embodiments, force in direction 620 alone is not enough to generate this disengagement, e.g., due to recess 604 being deep enough and/or upper edge 604A being long enough that interference with arm section 610 prevents it. In some such embodiments, however, disengagement can be achieved by exerting an arm-proximal directed force on lower member 604B (e.g., generally in the direction of arrow 621). Force sufficient to accomplish this is potentially only encountered during manufacturing operations, e.g., as part of packaging a suture clip into a delivery configuration, for example as described in relation to FIG. 1E, and/or because of the way that an overtube 113 impinges on anchor elements during withdrawal of the clip into the overtube 113. It should be noted, however, that irreversible expansion of the anchors may be undesirable, as it could prevent retrieval of a clip, e.g., in the case of an aborted implantation.

The configuration and changing shapes of anchor element 600 affect the way forces are responded to in its different configurations. For example, the collapsed delivery configuration of FIG. 6C may only be achieved by exerting a force in which it is generally not exposed to after deployment, unless deliberately; e.g., to remove it. It is an option, in some embodiments to revert an expanded anchor to the collapsed delivery configuration by withdrawing it back within the walls of an overtube 113; either the original delivery overtube 113, or a retrieval overtube 113.

Once elastic member 607 bends (with corresponding expansion of anchor element 600) to allow recess 604 to move to where it is fully engaged, forces exerted on elongated member 603, whether in the arm-distal direction (arrow 620) or in the anchor-proximal direction (arrow 622) may be blocked from causing deformations at least partially because upper edge 604A is not well oriented to slide out of engagement until tip 601 is already pushed far from the perpendicular (e.g., 30° or more; 45° or more). In effect, the engagement of recess 604 allows the compression strength of elongated member 603 to support elastic member 607 so that freedom of movement is confined to pivoting instead of sliding, unless a deliberate or unexpectedly great force is somehow brought to bear.

Conversely, elastic member 607 acts in part as a compression support against torque exerted on tip 602 in an arm-proximal direction (although under sufficient force it may buckle). Since lower member 604B interferes with recess 604 pulling out of engagement, elastic member 607 is furthermore constrained to buckle about its midpoint, rather than monotonically bend from its connection with member 605A, 605B, potentially increasing the amount of force needed to generate movement.

Attachment of elastic member 607 well above the base (e.g., at least ⅓ of the way to the tip 601 along the anchor-distal axis) also reduces the lever advantage for torque exerted on tip 601, compared to the lever advantage provided by rotation which would use the base as a pivot.

Described another way, the dual-base mounting configuration of anchor element 600 creates a triangular support structure which distorts only if one of the three sides can change length. Moreover, of those three sides:
A lower (anchor-proximal) portion of elongated element 603 is built rigidly. Although it may pivot somewhat, it is prevented from sliding (and shortening its side of the triangle) when recess 604 is engaged with arm section 610.
The span between the two bases (recess 604 and recess 606) is prevented from expanding under tension, because arm section 610 itself is rigid. Compression forces sufficient to dislodge engagement are unlikely to occur accidentally, e.g., lower member 604B does not extend out far enough to offer a large mechanical advantage to anything which might catch on it or press against it.
Only elastic member 607, which defines the remaining side of the triangle, is configured to bend—this is what allows anchor member 600 to be placed in a collapsed delivery configuration, and to transition to an expanded configuration upon deployment. However, once the other two sides of the triangle are in their own effectively rigid configurations, elastic member 607 can only distort the triangle (lengthen or shorten) by straightening or buckling about its middle. Because of its relatively rigid end mounting, this mode of bending involves compression at the ends and tension on the middle (on one longitudinal side) or vice versa (on its opposite longitudinal side). To this type of transformation, elastic member 607's shape is more resistant than it is to bending with uniformly one-sided compression/tension end-to-end from its basal connection to members 605A, 605B. The latter is a bending mode available to elastic member 607, e.g., when converting from the collapsed delivery configuration to the expanded configuration.

Reference is now made to FIGS. 7A-7C, which schematically represent a variation of a sprung tissue anchoring mechanism, according to some embodiments of the present disclosure.

FIG. 7A shows anchor element 700 alone (in a collapsed delivery configuration as though held compressed), including tip 701, barb 702, elongated member 703 recess 704, elastic member 707, recess 708; and upper member 705A and lower member 705B defining recess 708. These generally correspond in arrangement and function to what is described in relation to anchor element 700, except in certain specifics for members 705A, 705B and recess 708, as will be described.

FIG. 7B shows a distal arm section 710, including receiving aperture 713 with distal wall 711 and proximal wall 712.

FIG. 7C shows the components 700, 710 assembled. Compared to recess 608 of FIGS. 6A-6D, upper and lower members 705A, 705B leave a more open-sided recess 708. The more open-sided recess can engage directly with proximal wall 712, obviating the use of a bar 614 as described in relation to FIG. 6D. This provides is a potential advantage for manufacture of receiving aperture 713 itself, and/or for fitting anchor element 700 into receiving aperture 713. It otherwise operates as described for the anchoring mechanism of FIGS. 6A-6D. It is noted that the reliable engagement of recess 708 with distal arm section 710 is reliant on constant mutual support from elongated member 703 and/or recess 704 to maintain it in position. In comparison, recess 606, once installed, maintains its grasp on bar 614 with or without pressure assistance from recess 604 and/or elongated member 603.

It should be noted that there is in general no absolute requirement that the orientation of an anchor element 700, 600 along the arm-distal to arm proximal axis be as shown in FIGS. 6A-7C; it is optionally reversed so that the elongated member folds down proximally (e.g., toward an acute angle) instead of distally (e.g., toward an obtuse angle). However, the orientation shown in these figures has a potential advantage for initial packaging and/or reversibility of deployment—the distal direction of folding is the same as the direction of force which withdrawal into an overtube 113 would tend to exert. Folding in the other direction has a potential advantage for reducing a chance of “catching” on the sides of the overtube during deployment, since the sharp points 601, 701 are oriented away from their direction of travel in this case.

Reference is now made to FIGS. 8A-8B, which show one branched arm 820 of a suturing clip in a partially collapsed configuration (FIG. 8A) and a partially deployed configuration (FIG. 8B).

Core 102 is shown in cross-section; it connects to strut 104A which gives rise to several branching arms 104. Anchors elements 810 as illustrated correspond generally to the design of FIGS. 7A-7C, but should be taken as examples optionally substituted with another anchor element design, e.g., the design of FIGS. 6A-6C.

FIG. 8A shows anchor elements 810 “collapsed”—that is, confined by forces acting upon them to lay at an oblique angle to the arms 104 upon which they are mounted. Arms 104 are also represented as though partially confined. In FIG. 8B, confinement is reduced or removed, allowing mounting element 810 to spring out to their deployed expanded configuration.

### Suturing Clip Delivery Attachment Mechanisms

Reference is now made to FIGS. 9A-9E, which schematically illustrate a mechanism which provides releasable attachment between a suture clip 100 and its delivery mechanism 110, according to some embodiments of the present disclosure.

FIG. 9A shows a close view of core 102, including a portion of an arm strut 104A. Mounting plate 902 is partially held in position by brackets 904 within a cylindrical (or otherwise shaped) body 901 of core 102. In some embodiments, brackets 904 conversely are held in place by mounting plate 902, due to mutual interference between the two components. In some embodiments, brackets 904 and brackets 905 are both extensions of strut 104A (for example, formed from a single blank, optionally a blank cut from sheet stock of, e.g., nitinol). Aperture 903 is optionally used in assembly as follows: with brackets 904 inserted into body 901, mounting plate 902 is inserted over them by sliding it into the lumen of body 901 through aperture 903. Optionally, part of mounting plate 903 remains within aperture 903, which provides it support in opposition to pressure from bracket 904. Additionally or alternatively, the interior of body 901 comprises further element (e.g., shelf-like cutouts) which provide this support.

Operational details of the releasable attachment mechanism are shown in FIGS. 9B-9E. FIG. 9B illustrates a gripper 920, comprising a hollow body 921, and arms 822 (two, in some embodiments, but another number is optionally provided, for example, 3, 4, or 5). Each arm 922 bears a section with a narrowing 924, wider on either side, e.g., back proximally along the main body of arm 922, and at distal widening 923. Although shown straight, arms 922 are biased to bend radially inward when unconstrained. FIG. 9C shows mounting plate 902 alone (although it is normally mounted as described in relation to FIG. 9A), including aperture 910 keyed with cutouts 911, one for each arm 922 which is to engage with it.

In FIG. 9D, a locking element 930 is shown which has been advanced out of the hollow in gripper 920, forcing arms 922 apart and into engagement with the narrowings 924 fitted within the cutouts 911 of plate 902. In some embodiments, locking element extends all the way through aperture 910 of mounting plate 902 (and aperture 910 is sized to allow this), although locking is optionally achieved simply by bringing it up close enough to mounting plate 902 that narrowings 924 are immobilized in place.

Since arms 922 are wider on either side of each narrowing 924, plate 902 (and the rest of the suture clip 100 to which it is attached) is locked onto gripper 920 as long as locking element 930 remains in place.

In FIG. 9E, locking element 930 has been removed (e.g., withdrawn into the hollow of hollow body 921). Arms 922 are freed to spring radially inward according to their preset bias, releasing mounting plate 902.

It should be noted that the release process is optionally performed in reverse as a capture process: from the configuration of FIG. 9E to the configuration of FIG. 9D. This reversed process is optionally performed in vivo as part of the retrieval of an already deployed surgical clip.

### Arm Anchors

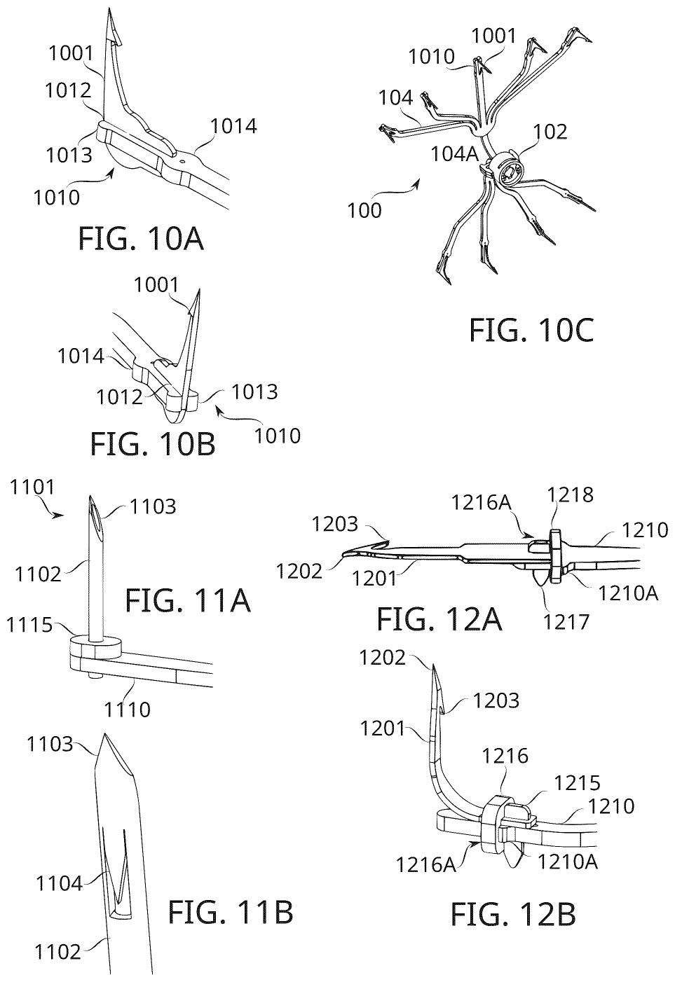

Reference is now made to FIGS. 10A-10C, which schematically illustrate a fixed-shape two-part anchor mechanism, according to some embodiments of the present disclosure.

Anchor element 1001, in some embodiments, comprises a piece cut from a thin sheet stock, the sheet stock thickness being smaller than a width of a distal arm distal section 1010 into which a slot 1012 has been cut to accommodate the thickness of anchor element 1001. Anchor element 1001 is optionally secured in place by welding (e.g., laser microwelding) and/or glue. Optionally, interlocking shapes are cut into element 1001 and/or slot 1012; optionally one or more pins passed into apertures of anchor element 1001 and/or slot 1012 are used to help secure element 1001 in place. Optionally, the slot 1012 is strengthened by increased material thickness of the arm distal section, for example, at protuberances 1013 and/or 1014.

FIG. 10A-10B show the same anchor-and-arm configuration from two different viewing angles. FIG. 10C shows the same anchor-and-arm configuration in the context of a complete suturing clip 100, including core 102, strut 104A, and arms 104.

Reference is now made to FIGS. 11A-11B, which schematically illustrate a needle-based anchor mechanism, according to some embodiments of the present disclosure.

Anchor element 1101, in some embodiments, comprises a tubular member 1102, optionally comprising a bevel 1103 at its distal end, and mounted to a distal arm section 1110 through an aperture 1113 sized to receive a cross-section of the tubular member 1002. Attachment, in some embodiments, is secured by welding. In some embodiments, a base plate 1115 is added to the mounting area to reinforce the strength of attachment of anchor element 1101 to distal arm section 1110. For example, base plate 1115 is also provided with a hole through which tubular member 1102 inserts. In some embodiments, base plate 1115 is welded to both tubular member 1102 and to arm distal section 1110.

In some embodiments (detail view of FIG. 11B), tubular member 1102 is provided with a barb 1104 by using laser cutting to free a flap of wall material. The barb 1104 can be bent outward so that it functions to assist anchoring.

Reference is now made to FIGS. 12A-12B, which schematically illustrate an anchoring member 1201 secured to an arm distal section 1210 by interlocking fasteners, according to some embodiments of the present disclosure. FIG. 12A shows an example which is flattened, e.g., as it would be in a collapsed delivery configuration constrained by surrounding elements, and FIG. 12B shows an example in a deployed configuration. The two examples are somewhat different in the arrangement of their interlocking fasteners.

Anchoring member 1201, in some embodiments, is made of a superelastic shape-memory alloy, biased to assume the approximately right-angle curved shape of FIG. 12B, but sufficiently flexible to lie flat when packaged for delivery. The sheet stock from which anchoring member 1201 is constructed is optionally about 2-3× thinner than material of distal arm section 1210; e.g., having a thickness of about 500 μm-1 mm. This potentially allows a smaller radius of curvature than is practical for the material of arm distal section 1210 itself. Potentially, the thinner material also more easily penetrates tissue. As shown, anchoring member 1201 has been provided with a sharpened tip 1202 and barb 1203.

Shown in FIG. 12B is an interlocking fastener comprising retaining pin 1215 and plate 1216. Plate 1216 comprises an aperture 1216A which is sized to allow it to slide onto arm distal section 1210, up to the position of stops 1210A. Anchoring member 1201 inserts also through aperture 1216A, e.g., within a keyed section on one side of aperture 1216A. Anchoring member 1201 may be narrower along its length so that once seated to its keyed section, it cannot be pulled out. Retaining pin 1215 inserts through apertures in both anchoring member 1201 and arm distal section 1210. Retaining pin 1215 may itself be held in place by, e.g., welding, plastic deformation (e.g, bending over), and/or additional interlocking interactions (e.g., it may be shaped to snap into place).

The arrangement of FIG. 12A reverses the proximal-to-distal placement of retaining pin 1217 and plate 1218, but otherwise uses a similar arrangement of interlocking interferences and optional additional attachment to secure anchoring member 1201 in place.

### Pre-Deployment Positioning

Reference is now made to FIG. 13, which schematically represents internal structure of an overtube 113 of a distal tip 112 of a catheter for delivering a suturing clip 101, according to some embodiments of the present disclosure.

In some embodiments, overtube 113 includes features to promote visualization of delivery system 110 during positioning and/or deployment. In some embodiments, proper pre-deployment positioning is achieved by focusing on the control of three degrees of freedom: a correct predetermined rotational orientation of the suturing clip 101 (not shown in FIG. 13), centering of the delivery system 110 relative to the LAA ostium 3 along the anterior/posterior axis, and advancing of the delivery system 110 by about 1-8 mm past the circumflex coronary artery. In some embodiments, these degrees of freedom are addressed in sequence, e.g., in the sequence just listed. In some embodiments, setting of each degree of freedom is performed under imaging observation, optionally using different view angles and/or imaging modalities, as appropriate.

For example, in some embodiments:
The orientation of the suturing clip 101 is set under fluoroscopic visualization;
Anterior/posterior centering of the delivery system 110 is set using ultrasound imaging, optionally 3-D imaging; and
Pre-deployment insertion depth of the delivery system 110 is set using planar ultrasound imaging that includes a plane of the circumflex coronary artery.

In FIG. 13, an overtube 113 is shown which includes features of its construction which potentially assist in the types of visualization just mentioned, as well as with safe (sufficiently strong) containment of suturing clip 101 (a suturing clip is not shown in FIG. 13, but when present within overtube 113, it generally occupies the lumenal region exposed in cross section).

In some embodiments, a distal region of overtube 113 is multilayered. An inner layer 1312 may comprise metal, for example, stainless steel. This may be provided to provide sufficient strength so that it acts as a cylindrical capsule to contain the spring forces of suturing clip 100, 101 in its collapsed position. The length of this region may be enough to encapsulate all of suturing clip 100, 101, or a portion of it, e.g., the portion along which outward forces are the greatest, and/or the portion containing sharp elements such as anchors.

Optional layer 1315, in some embodiments, comprises a braided sleeve. The complex structure of the braided construction, with many small surfaces oriented in many different directions, may be relied on to provide echogenicity to the distal tip, potentially enhancing visualization of tip position under ultrasound imaging. Optionally, echogenicity is otherwise provided, e.g., by roughening the outer surface of the inner layer 1312. Inner layer 1312 may be roughened, for example, by providing it with ridges, grooves, slots and/or another structure to enhance its echogenicity. Layer 1315 may extend along all of inner layer 1312, along a portion of it, and/or beyond it. It may be continuous or discontinuous.

Ring 1314, in some embodiments, comprises a radiopaque material such as gold, tungsten, tantalum, or another metal. This potentially enhances visualization of tip position under X-ray (fluoroscopic) imaging.

Accordingly, overtube 113 is optionally provided with both echogenicity (e.g., via inner tube 1312 and/or layer 1315) to promote ultrasound visualization, and with radiopacity (e.g., via radiopaque ring 1314) to promote fluoroscopic visualization.

Liner 1313, in some embodiments, comprises a low-friction polymer such as PTFE. This potentially helps to reduce resistance to manipulation to advance/retract suturing clip 101. Optionally, liner 1313 is constructed of a different material than outer layer 1311, which may comprise, e.g., a different polymer; for example, Pebax.

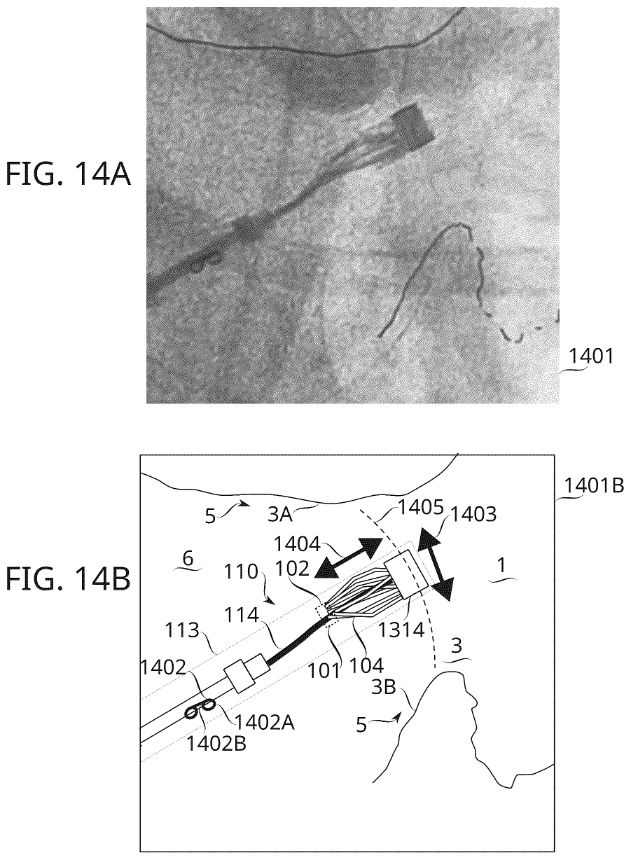

Reference is now made to FIGS. 14A-14B, which show, respectively, a fluoroscopic image 1401 (FIG. 14A) and corresponding image trace-over 1401B (FIG. 14B) of a suturing clip 101 and delivery system 110 in situ as it passes through an LAA ostium 3, according to some embodiments of the present disclosure. Due to radiolucency, not all of suturing clip 101 is clearly visible in fluoroscopic image 1401. The arms 104 are generally discernable. The approximate position of core 102 is indicated by dotted lines in FIG. 14B.

Double-headed arrow 1404 represents distal/proximal directions of movement of delivery system 110 as it is advanced/retracted through LAA ostium 3 into the LAA 1 itself, from the left atrium 6. As shown, the atrial wall 5 gives way to the anterior wall 3A and posterior wall 3B of the LAA ostium 3. Partial wall outlines including anterior wall 3A and posterior wall 3B are marked in image 1401 as dark overlay lines. During a procedure, their positions may be determined, e.g., by noting outlines transiently revealed after an injection of radiopaque marker dye.

Double-headed arrow 1403 represents available directions of movement of a distal tip of delivery system 110 along arc 1405 as it is steered. Radiopaque marker ring 1314 is part of overtube 113, the remainder of which is drawn in dotted lines in FIG. 14B to indicate the approximate position of its radiolucent (transparent to X-rays) outline.

In some embodiments, suture clip 101 has a preferred orientation of deployment. This orientation is selected so that the eventual line of closure of LAA ostium 103 extends in a predetermined direction (e.g., generally parallel to the plane of the mitral valve). The preferred orientation also helps ensure that anchor engagement is to tissue in known portions of the LAA and/or LAA ostium. To achieve an intended orientation, delivery mount 114 together with suturing clip 101 may be rotated within overtube 113. Optionally, they are rotated together with overtube 113.

Moreover, in some embodiments, steering results in movement of the distal tip of delivery system 110 through a planar region. For clarity of navigation, it is a potential advantage for this planar region to be aligned perpendicular to the direction of the image view. This helps assure that steering movements achieve intended centering of the device along the anterior/posterior axis, preferably without concomitant displacement along the superior/inferior axis. Centering may also reduce a likelihood of accidentally dislodging anchored material such as a pre-existing blood clot.

In some embodiments, radiopaque marker 1402 is provided on a portion of delivery system 110, and shaped to indicate the present orientation of suture clip 101 and/or the steering mechanism. For example, radiopaque marker 1402 is provided with one or more loops 1402A. The loops 1402A are optionally arranged to lie generally within the steering plane, so that they assume their most open-centered (e.g., most nearly circular) appearance when the plane of steering is oriented perpendicular to the viewing direction of the image. Even when oriented correctly, there may be deviation from apparent circularity due to foreshortening in another direction, e.g., axial foreshortening when the proximal-distal axis of the delivery system 110 is not perpendicular to the direction of viewing. In the example, two loops 1402A are connected by a backbone 1402B, so that radiopaque marker 1402 assumes a “B”-like shape when viewed perpendicular to the steering plane. The position of backbone 1402B relative to loops 1402A may be used to distinguish orientations separated by 180 degrees of rotation. The apparent relative axial offset of the loops 1402A may assist in determining the degree of axial foreshortening.

In some embodiments, fluoroscopic visualization is used to help ensure that the suturing clip 101 is correctly oriented. After this, visualization optionally switches to ultrasound-based imaging, which has potential advantages for providing live 3-D imaging, and/or discontinuing the use of ionizing radiation for imaging.

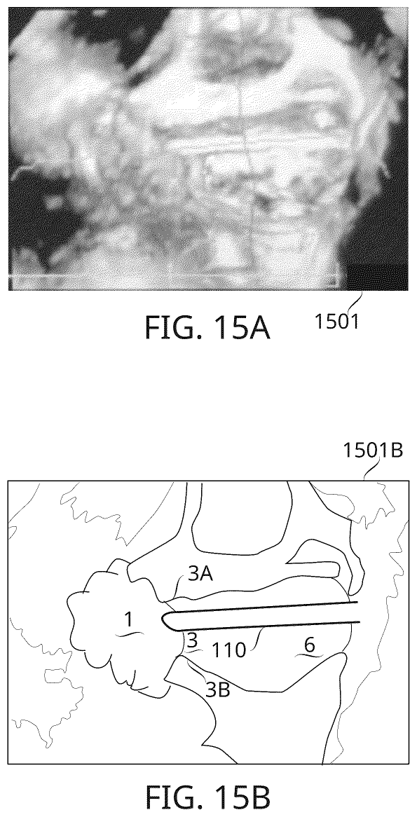

Reference is now made to FIGS. 15A-15B, which show, respectively, a 3-D (three dimensional) ultrasound image 1501 (FIG. 15A) and corresponding image trace-over 1501B (FIG. 15B) of a delivery system 110 for a suturing clip in situ as it passes into an LAA ostium 3, according to some embodiments of the present disclosure. Ultrasound image 1501, in some embodiments, comprises ultrasound echo visualization performed by trans-esophageal echocardiography (TEE), and/or by ICE (intra-cardiac echocardiography).

In the orientation shown, delivery system 110 is advancing leftward through the left atrium 6 (on the right) into the LAA ostium 3, and protruding a short distance into the LAA 1 itself. The anterior wall 3A and posterior wall 3B of the LAA ostium 3 are shown as before. The image shows the LAA ostium 3 cross-sectioned in 3-D, e.g., sliced from top to bottom and left to right through the anterior and posterior walls 3A, 3B to reveal the cross-sections outlined with dark contour lines in FIG. 15B.

Optionally, delivery system 110 is advanced through LAA ostium 3 under ultrasound imaging, while maintaining it about equally spaced from the anterior and posterior walls 3A, 3B. This centering helps to ensure proper placement of the device for deployment. Optionally, the device is allowed to self-center along a ventral-dorsal axis of the LAA ostium 3, e.g., an axis corresponding generally to a direction perpendicular to the plane of FIGS. 15A-15B. In some embodiments, self-centering occurs since, at maximum spread, the deploying arms 104 of the suturing clip 101 extend across all or nearly all of the ventral-dorsal height of the LAA 1 between a “floor” near to the plane of the mitral valve, and a “roof” opposite it. Along the orthogonal (anterior-posterior) axis, positioning is potentially less constrained by the deployment space, i.e., the LAA 1 may be about as “tall” as the floor-to-roof reach of the expanded suturing clip 101, but significantly “wider” (in the orthogonal direction) than the side-to-side reach of the expanded suturing clip 101,

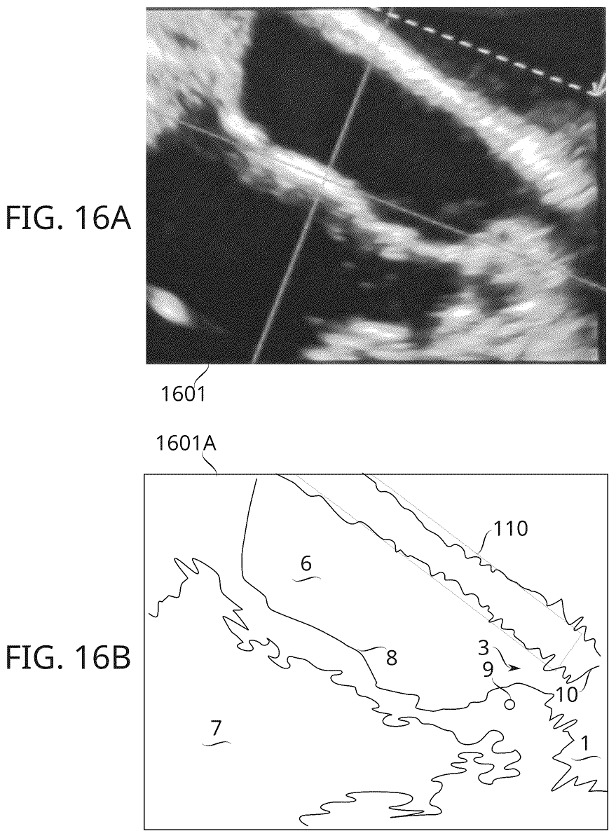

Reference is now made to FIGS. 16A-16B, which show, respectively, a 2-D (two-dimensional) ultrasound image 1601 (FIG. 16A) and corresponding image trace-over 1601A (FIG. 16B) of a delivery system 110 for a suturing clip in situ as it passes through an LAA ostium 3, according to some embodiments of the present disclosure. In this case, the LAA 1 is shown to the right, and the left atrium 6 to the left, separated by LAA ostium 3. Delivery system 110 is advancing left-to-right from the direction of the atrial septum, above mitral valve 8 and left ventricle 7.

As a visualization landmark, the circumflex coronary artery 9 may be used. In a suitably arranged ultrasound cross-section, e.g., as shown, the circumflex coronary artery 9 may appear as a dark (low echogenicity) hole in tissue structures extending toward the LAA 1 from the mitral valve 8. Advance of the distal tip of delivery system 110 past this landmark may be, e.g., to a depth of about 1-8 mm before deployment of the suturing clip it contains is initiated.

As visualized, a distal tip of delivery system 110 is shown somewhat distended, because it is currently expressing a droplet of a fluid echo contrast agent 10. This helps to delineate its distal tip to provide ultrasound visualization. In some embodiments, delivery system 110 comprises a lumen dedicated to transportation of contrast agent 10, for example, a dedicated tube extending along the lumen of overtube 13.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A self-expanding tissue anchoring mechanism configured to deploy from an arm of an implantable device, the anchoring mechanism comprising:
an arm, including an anchor-mounting portion;
an anchoring element having a sharp distal end, and a proximal end engaged with the anchor-mounting portion;
wherein, together, the proximal end and anchor-mounting portion form an assembly, the assembly having an elastic member which relaxes to interconvert the assembly between a first shape and a second shape; and
the first shape is collapsed to position a distal portion of the sharp distal end of the anchoring element relatively close to a longitudinal axis of the arm, compared to the second shape.

2. The anchoring mechanism of claim 1, wherein the elastic member is tensioned in the first shape, and relaxed in the second shape.

3. The anchoring mechanism of claim 1, wherein interconversion between the first shape and the second shape moves the distal portion of the sharp distal end of the anchoring element longitudinally along the longitudinal axis, and further away from the longitudinal axis.

4. The anchoring mechanism of claim 1, wherein the elastic member comprises a spring formed into the anchoring element;
wherein the anchoring element comprises a piece cut from sheet stock, and the elastic member is a part of the piece.

5. (canceled)

6. The anchoring mechanism of claim 1, wherein the anchoring element and the anchor-mounting portion comprise separate interlocking pieces.

7. The anchoring mechanism of claim 1, wherein the anchoring element comprises one or more of:
a. a base, attached to the anchor-mounting portion of the arm;
b. the elastic member, extending between the sharp distal end and the base;
c. a base, attached in a plurality of separate base regions to the anchor-mounting portion of the arm;
wherein the elastic member joins the sharp distal end to one of the base regions; and
an elongated member joining the sharp distal end to another of the base regions, bypassing the elastic member.

8. (canceled)

9. The anchoring mechanism of claim 1, wherein the implantable device is a suturing clip comprising a plurality of arms extending from a core, each provided with a corresponding anchor element.

10. The anchoring mechanism of claim 1, wherein the sharp distal end is held away from the anchor mounting portion of the arm by an elongated member, wherein a longitudinal axis of the elongated member extends away from a longitudinal axis of the arm at an approximate right angle in the second shape, and at an oblique angle in the first shape which is at least 30° different than the approximate right angle shape; and
wherein the oblique angle in the first shape is at least 45° different than the approximate right angle shape.

11. (canceled)

12. The anchoring mechanism of claim 1, wherein the elastic member of the assembly is part of the proximal end of the anchoring element; and
wherein the elastic member terminates in a recess shaped to engage with the anchor-mounting portion.

13. (canceled)

14. The anchoring mechanism of claim 1, wherein the elastic member of the assembly is part of the anchor-mounting portion; and
wherein the elastic member terminates in a recess shaped to engage with the anchoring element.

15. (canceled)

16. A self-expanding tissue anchoring mechanism configured to deploy from an arm of an implantable device, the anchoring mechanism comprising:
an anchoring element terminating in a sharp distal end and attached, through a plurality of base regions on its proximal side, to an anchor-mounting portion of the arm;
wherein the sharp distal end is joined to a first of the plurality of base regions through an elastic member, and separately to a second of the plurality of base regions through a separate elongated member.

17. The self-expanding tissue anchoring mechanism of claim 16, wherein the elastic member bends, moving the sharp distal end from a collapsed position nearer to a longitudinal axis of the anchor-mounting portion of the arm, to an expanded position further from the longitudinal axis.

18. The self-expanding tissue anchoring mechanism of claim 17, wherein the second of the base regions comprises an open-sided recess, the recess being engaged with the anchor-mounting portion of the arm when the sharp distal end is in the expanded position, and disengaged when the sharp distal end is in the collapsed position.

19. The self-expanding tissue anchoring mechanism of claim 18, wherein the open-sided recess is positioned near a base end of an elongated member, and as the sharp distal end moves from the collapsed position to the expanded position, the elongated member slides along the anchor-mounting portion of the arm until the open-sided recess engages with the anchor-mounting portion.

20. The self-expanding tissue anchoring mechanism of claim 16, wherein the first of the plurality of base regions comprises a pair of members which grasp a portion of the anchor-mounting portion of the arm from on opposite sides of the anchor-mounting portion.

21. The self-expanding tissue anchoring mechanism of claim 20, wherein the pair of members clasp around a bar to define a slot between them through which the bar is passed to snap-fit the anchoring element to the anchor-mounting portion of the arm.

22-25. (canceled)

26. A method of expanding an anchoring member from an arm of a suturing clip, the method comprising:
providing an anchoring member attached to a portion of the arm, and confined within a lumen of an overtube, wherein the anchoring member comprises an elongated member:
contacting the arm at a proximal end of the elongated member,
terminating in a distal sharpened end, and
constrained by confinement of the anchoring member to an oblique angle with respect to a longitudinal axis of the portion of the arm;
releasing the anchoring member from confinement within the lumen; and
as the anchoring member is released from confinement within the lumen:
sliding the elongated member along the arm until a recess of the elongated member reaches the arm and grasps it, and
pivoting the elongated member around a region of contact between the arm and the recess until the elongated member reaches an approximate right angle with respect to the longitudinal axis.

27. The method of claim 26, wherein the sliding and the pivoting are driven by tension released from a spring member as the anchoring member is released from confinement within the lumen;
wherein the sliding advances the elongated member through an aperture of the arm; and
wherein the pivoting rotates the recess around a wall of the aperture which is within the grasp of the recess.

28-29. (canceled)

30. A method of collapsing an anchoring member on an arm of a suturing clip, the method comprising:
providing an anchoring member attached to a portion of the arm protruding from a lumen of an overtube, wherein the anchoring member comprises an elongated member:
having a recess of the anchoring member located at a proximal end of the elongated member where the recess is positioned to grasp a region of the arm, and
terminating in a distal sharpened end;
withdrawing the arm and the attached anchoring member into the lumen; and
as the anchoring member is withdrawn into lumen:
under contact force exerted by the overtube, pivoting the elongated member around a region of contact between the arm and the recess until the recess releases from engagement with the region of the arm, and
sliding the elongated member further along the arm;
thereby flattening the elongated member into a more oblique angle with respect to a longitudinal axis of the portion of the arm to which it attaches.

31. The method of claim 30, wherein the sliding and the pivoting develop tension in a spring member of the anchoring member; and
wherein the anchoring member is embedded in tissue, and comprising extracting the anchoring member from tissue of less than 5 mm thickness without leaving behind an open hole.

32-35. (canceled)
